(19) United States
(12) Patent Application Publication
(10) Pub. No.: US 2004/0057962 A1
(43) Pub. Date: Mar. 25, 2004

(54) Immunogenic complex

(76) Inventor: Benedikt Timmerman, Toulouse (FR)

Correspondence Address: HELLER EHRMAN WHITE & MCAULIFFE LLP, 1666 K STREET,NW, SUITE 300, WASHINGTON, DC, 20006, US

(21) Appl. No.: 10/250,668

(22) PCT Filed: Jan. 4, 2002
(86) PCT No.: PCT/IB02/00738

(30) Foreign Application Priority Data
Jan. 6, 2001 (GB) 0100758.2

(51) Int. Cl. A61K 39/02; A61K 39/002; A61K 48/00

(52) U.S. Cl. 424/190.1; 514/44; 424/191.1

(57) ABSTRACT

The present invention relates to an immunogenic complex comprising a ribosomal complex of a microbe and a polynucleotide molecule encoding an antigen originating, derived from, or deduced from a microbe or a virus. The Ribosomal Complex is composed of the subunits of ribosomes (50 S and 30 S subunits in bacteria and 60 S and 40 S subunits in eucaryotes), the ribosomal subunits generally retaining sufficient integrity to preserve substantially the double-stranded nature of the large r-RNA's (16 S and 23S in bacteria; 18S and 28S in eukaryotic cytosol) contained in the ribosomal subunits.

### FIELD OF THE INVENTION

This invention relates to an Immunogenic Complex, method of preparation thereof and Pharmaceutical compositions containing the same, useful for disease control.

### BACKGROUND OF THE INVENTION

Primary entry ports for pathogens are the mucosal surfaces of respiratory tract, gastrointestinal tract, eye, ear and nasal tracts and genital tracts. Higher vertebrates have developed efficient immune defense mechanisms, such as the Mucosal Immune System to halt colonising microbes as well as to eliminate infecting pathogens. The mucosal immune responses are functionally distinct from systemic immune responses, and are stimulated by antigen presentation within specialised mucosa associated lymphoid inductor tissues such as the tonsils, oropharyngeal Waldeyer's ring or intestinal Peyer's patches and other M cell or dendritic cell containing sites.

Viruses attacking the respiratory tract such as human rhinoviruses, influenza viruses, parainfluenza viruses, adenovirus and respiratory syncytial virus can be strongly associated with subsequent life-threatening secondary infections by bacterial pathogens such as Haemophilus influenzae, Streptococcus pneumoiniae, Staphylococcus aureus, Klebsiella pneumoniae, Neisseria meningitidis and Bordetella pertussis. Similar opportunistic interactions between viral and subsequent bacterial infections are suspected in gastro-intestinal diseases. In addition, symbiotic interactions amongst bacteria also occur, as has been demonstrated for bacterial pathogens associated with periodontal disease.

The immune system of vertebrates consists of several complementary components: innate anti-microbial agents, the complement system, the humoral and cellular immune systems. The latter two are best characterised and subject to vaccination strategies. Humoral immunity is implemented by antibodies (immunoglobulins) which are proteins that are produced and secreted by special B lymphocytes (B cells). Antibodies subsequently circulate systemically through the body via body fluids such as the blood and can directly recognise an antigen, bind to it, deactivate it (and the pathogen to which it belongs) or activate other cells of the immune system to destroy the invader. Cellular immunity is mediated by a special class of lymphoid cells called Cytotoxic T Lymphocytes (CTL) or T cells. These cells recognise altered host cells and kill them by inflicting membrane damage and releasing signals for apoptotic death (suicide) of the infected cell and in the process also eliminating the pathogen.

An immense body of literature is available on microbial Antigens of most important bacteria, fungi, protozoa and viruses of man and life-stock. Pathogen-neutralising antibodies are mostly directed against surface components of the pathogen such as viral capsid structures in non-enveloped viruses and envelope (glyco)proteins in enveloped viruses, cell-membrane or cell wall macro-molecules/structures for bacteria, fungi and protozoa. Many critical antibody binding sites are complex and conformation-dependent. For example, alternative glycosilation patterns in highly variable parts of surface antigens may allow viruses to induce strong humoral host responses to immuno-dominant epitopes, not infrequently comprising spatially related amino acid residues of more than one protein and subsequently, through alternative glycosilation, to mask them and escape the majority of neutralising antibodies. Modelling of such epitopes is still experimental. In addition, pathogen populations often circumvent prior immunisation of the host due to their extraordinary multiplication ability and relatively high mutation ratio which leads to sub-populations carrying altered surface epitopes which are no longer recognised by the host. These features explain in part the cause for the relatively few vaccines which provide long-lasting protection against specific diseases.

Most vaccines on the market and under development are directed towards the induction of systemic antibodies and, to a lesser degree, cell-mediated immune response. A wide variety of simple and combination vaccines have been developed which can be classified as inactivated pathogen vaccines, attenuated live vaccines, purified or recombinant subunit vaccines and more recently polynucleotide-based vaccines.

None of the vaccines developed so far induce effective mucosal, humoral and cellular immune responses which safely protect or treat the host against diseases caused by concomitant or subsequent combinations of pathogens, such as is the case in the above mentioned viral/bacterial associations and periodontal disease. Consequently, there is a strong need for improved vaccines which are capable of safely inducing broad and effective immune responses against multiple strains or stereotypes of a pathogen or against multiple pathogens. Background information on critical aspects of the current invention follow below.

Delivery of vaccines via the mucosal immune system for the induction of immunity against respiratory diseases is desirable as these mucosa represent the first barrier for pathogens. In addition, the mucosal tissues contain large quantities of immuno-reactive cells, many times more than the lymphoid cells of the bone marrow, spleen, thymus and lymph nodes together. A mucosally induced B cell response leads to massive production of antigen-inactivating immunoglobulin A (IgA) molecules, which are targeted and secreted at appropriate effector mucosal tissues. The gastrointestinal, urogenital and respiratory tracts of vertebrate animals are covered with mucus on the surface of mostly a single layer of epithelial cells and under which the mucosal immune tissues lie.

Most immuno-dominant epitopes of microbial or viral antigens are exposed on the surface of the pathogens and can have a variety of functions which are often related to either the pathogen's structure, host (cell) range or virulence. More recently, it has been shown with polynucleotide vaccines that also internal factors of pathogens can be immunogenic and can be useful for control of pathogens which go through an intracellular stage in their infection cycle. Adhesins are microbial cell-surface components which mediate tight adhesion of the pathogen to its host cell, or to the extra-cellular matrix macromolecules, which embed host cells. Adhesion commonly induces the secretion of pathogen-derived factors which impair host defence. Adhesins are at least partially surface exposed and play a crucial and early role in virulence of the pathogen. Unfortunately, adhesin epitopes on pathogen surfaces are often not accessible or insufficiently immunogenic to cause immunity to their host.

Youmans and Youmans were the first to produce a protective vaccine, based on extracts of ribosomes (Youmans A. S. and Youmans G. P., 1965). Since then experimental vaccines incorporating ribosomal preparations from different bacterial, fungal and protozoan microorganisms have been described. Few of these vaccines have made it beyond the laboratory experiment, probably because the active principles of ribosomal extracts were commonly lost upon preparation, leading to irregular and even contradictory results.

The Immunogenic complex, subject of present invention, addresses many of the limitations of current vaccines such as multi-epitope, multi serotype or multi-strain recognition, stimulation of strong mucosal IgA response in addition to the more traditional humoral and cellular immune responses, usefulness in combination vaccines for management of multi-pathogen diseases and safety.

### SUMMARY OF THE INVENTION

The invention provides an Immunogenic Complex comprising Ribosomal Complex of Microbes and Polynucleotide Molecules encoding Antigen from, derived from, or deduced from Microbes or viruses. The invention also provides for methods to produce said Immunogenic Complex, to couple Ribosomal Complex to Polynucleotide Molecule and to package Immunogenic Complex in delivery systems.

The unique and deliberate combination of Ribosomal Complex and Antigen-encoding Polynucleotide Molecule in the Immunogenic Complex is surprisingly superior in immune induction and immune protection against targeted Microbes and viruses than the components alone and constitutes active ingredients of superior prophylactic or therapeutic vaccines for the management of diseases of animals including humans.

The present invention thus provides an Immunogenic Complex comprising at least one Ribosomal Complex and at least one Polynucleotide Molecule encoding Antigen of a Microbe. The invention also provides an Immunogenic Complex comprising at least one Ribosomal Complex and at least one Polynucleotide Molecule encoding Antigen, wherein Ribosomal Complex is from a Microbe and Polynucleotide encodes Antigen of virus. In a preferred Immunogenic Complex according to the invention, a Ribosomal Complex comprises complexes which originate from multiple Microbe species. Optionally, an Immunogenic Complex according to the invention, Polynucleotide Molecules encodes multiple Antigens.

Preferably, a Ribosomal Complex contains the large and small subunits of ribosomes in particulate form. Preferably, a Ribosomal Complex that carries minor fractions of microbial cellular membrane or cell wall components, and more preferably, a Ribosomal Complex retains sufficient integrity to largely preserve the double-stranded nature of the large r-RNA's contained in the subunits of ribosomes.

In a preferred Immunogenic Complex according to the invention, a Polynucleotide Molecule is a DNA molecule that comprises a DNA transcription unit that encodes an Antigen, said DNA transcription unit operatively linked to regulatory sequences which control the expression of the said DNA transcription unit. In selected embodiments, the regulatory sequences comprises the human immunoglobulin gene control region, and/or the rabbit β-globin gene transcription terminator sequence. Preferably, according to the invention, the expression of the DNA transcription unit, that encodes an Antigen, in a host cell leads to production of a protein which is capable of inducing an immune response against said Antigen. Preferably the host cells are eucaryotic cells belonging to vertebrate animal groups aves, Pisces and mammalia, including humans.

By way of example, Immunogenic Complex may comprise a Ribosomal Complex and/or Polynucleotide Molecule prepared, derived or deduced from:

(a) a bacteria selected from the group consisting of: Actinobacillus actinomycetemcomitans, Bacille Calmette-Guérin, Bordetella pertussis, Campylobacter consisus, Campylobacter recta, Capnocytophaga sp., Chlamydia trachomatis, Eikenella corrodeus, Enterococcus sp., Escherichia coli, Eubacterium sp., Haemophilus influenzae, Klebsiella pneumoniae, Lactobacillus acidophilus, Listeria monocytogenes, Mycobacterium tuberculosis, Mycobacterium vaccae, Neisseria gonorrboeae, Neisseria meningitidis, Nocardia sp., Pasteurella multocida, Porphyromonas gingivalis, Prevotella intermedia, Pseudomonas aeruginosa, Rothia dentocarius, Salmonella typhi, Salmonella typhimurium, Serratia marcescens, Shigella dysenteriae, Streptococcus mutans, Streptococcus pneumoniae, Streptococcus pyogenes, Treponema denticola, Vibrio cholera, and Yersinia enterocolitica;

(b) a fungus selected from the group consisting of Candida albicans and Blastomyces dermatitidis; or

(c) a protozoa selected from the group consisting of Plasmodium falciparum, Leishmania sp, Trypanosoma cruzi, and Entamoeba histolitica.

In other aspects, a Polynucleotide Molecule is prepared, derived or deduced from virus selected from the group consisting of: Influenza virus; parainfluenza virus; rhinovirus; hepatitis A virus; hepatitis B virus; hepatitis C virus; apthovirus; coxsackievirus; Rubella virus; rotavirus; Denque virus; yellow fever virus; Japanese encephalitis virus; infectious bronchitis virus; Porcine transmissible gastroenteric virus; respiratory syncytial virus; Human immunodeficiency virus; papillomavirus; Herpes simplex virus; varicellovirus; Cytomegalovirus; variolavirus; Vacciniavirus; and suipoxvirus.

In preferred Immunogenic Complex according to the invention, the Ribosomal Complex and Polynucleotide Molecule are incorporated in a polymeric matrix, preferably a matrix comrpising or consisting of chitosan-EDTA Bowman-Birk Inhibitor conjugate. In other aspects, Ribosomal Complex and Polynucleotide Molecule are incorporated in microparticles. In preferred examples, the micro-particles used is carboxymethylethylcellulose (CMEC) coated poly[dl-lactide-coglycolide] (PLG).

In certain aspects, Immunogenic Complex according to the invention comprise Ribosomal Complex and Polynucleotide Molecule which are non-covalently coupled by ionic interactions. In other aspects, the Ribosomal Complex is covalenty conjugated to a polycation and the Polynucleotide Molecule is condensed onto said Ribosomal Complex-polycation conjugate by ionic interaction. An example of the polycation used for conjugation to the Ribosomal Complex is poly(L-lysine), preferably where the average chain length of poly (L-lysine) ranges between 200 and 400 monomers. Preferably, Immunogenic Complex according to the invention comprises Ribosomal Complex and Polynucleotide Molecule which are covalently coupled. By way of example, covalent coupling can be achieved by reaction of Ribosomal Complex with 2-iminothiolate followed by addition of Polynucleotide Molecule and mild ultraviolet irradiation, or by reduction of 5′thio-derivatized Polynucleotide Molecule via the freed thiol-group to maleimide-derivatized Ribosomal Complex.

Also encompassed is a Pharmaceutical composition for prevention and/or treatment of infectious disease caused by Microbes and/or viruses comprising Immunogenic Complex according to the invention, wherein the Immunogenic Complex is formulated as a pharmaceutically acceptable vaccine for administration to animals and/or humans. Preferably the Pharmaceutical composition is used in prophylactic vaccines against Microbes and viruses, or preferably as immuno-modulator in therapeutic agents. In futher preferred embodiments, the Pharmaceutical composition of the invention is used as a therapeutic vaccine to activate an immune response against Antigen expressed by the infectious Microbes and/or viruses during their established pathogenic phase.

Pharmaceutical composition according to the invention can advantageously be used to control whooping cough caused by Bordetella pertussis, wherein the Immunogenic Complex comprises Ribosomal Complex (IC) derived from B. pertussis, coupled to Polynucleotide Molecule (PNM) encoding the Adhesin filamentous hemaglutinin (FHA) of B. pertussis or any related protein or polypeptide derived from or corresponding to part of the fha gene product, which can still induce an antibody response to FHA. In another aspect, the Pharmaceutical composition according to the invention can be used to control whooping cough caused by Bordetella pertussis and respiratory tract infections caused by respiratory syncytial virus (RSV), wherein the Bacterio-viral Immunogenic Complex comprises RC derived from B. pertussis which is coupled, for a % fraction with a PNM that encodes FHA, or any related protein or polypeptide derived from or corresponding to part of FHA, which can still induce an antibody response to FHA, and for the remaining % fraction is coupled with a PNM that encodes the fusion (F) glycoprotein (Fgp) of RSV, or any related protein or polypeptide derived from or corresponding to part of Fgp, which can still induce an antibody response to Fgp.

In another aspect, a Pharmaceutical composition according to the invention can be used to control candidiasis, wherein the Heterologous Immunogenic Complex comprises RC derived from Candida albicans coupled to PNM encoding the Adhesin HWP1 of Candida albicans, or any related protein or polypeptide derived from or corresponding to part of HWP1, which can still induce an antibody response to HWP1.

In another aspect, a pharmaceutical composition according to the invention can be used to control salpingitis and/or urethritis and/or cervicitis and/or trachoma, comprising RC derived from C. albicans coupled to PNM encoding Antigen SK59 of Chamydia trachomatis, or any related protein or polypeptide derived from or corresponding to part of SK59 which can still induce an antibody response to SK59.

In a further aspect, a pharmaceutical composition according to the invention can be used to control candidiasis and salpingitis and/or urethritis and/or cervicitis and/or trachoma, comprising RC derived from Canidida albicans which is coupled, for a % fraction with a PNM that encodes HWP1 of C. albicans, or any related protein or polypeptide derived from or corresponding to part of HWP1, which can still induce an antibody response to HWP1, and for the remaining % fraction is coupled with a PNM that encodes the SK59 protein of Chlamydia trachomatis, or any related protein or polypeptide derived from or corresponding to part of SK59 which can still induce an antibody response to SK59.

Also envisioned is the use of the Immunogenic Complex or the pharmaceutical composition according to the invention in the preparation of a medicament for prophylaxis or treatment of infectious diseases in humans or in animals. Preferably the Immunogenic Complex or the pharmaceutical composition are used for prophylaxis or treatment of systemic infection and urogenital, buccal and/or ocular diseases in humans or in animals, preferably for prophylaxis or treatment of diseases caused by Candida sp. in humans or in animals; preferably for prophylaxis or treatment of diseases caused by Chlamydia sp. in humans or in animals; preferably for prophylaxis or treatment of respiratory diseases in humans or in animals; preferably for prophylaxis or treatment of diseases caused by Bordetella sp. in humans or in animals; or preferably for prophylaxis or treatment of diseases caused by respiratory syncytial virus in humans or in animals.

In further embodiments, the invention provides a method of treating infectious diseases in humans or animals, or of providing prophylaxis in respect to said diseases, comprising administrating to said humans or animals an effective amount of the Immunogenic Complex or the pharmaceutical composition of the invention. Preferably, the method involves treatment or prophylaxis of urogenital diseases. The method may advantageously comprise treatment or prophylaxis of diseases caused by Candida sp., including buccal, urogenital and systemic candidiasis; treatment or prophylaxis of diseases caused by Chlamydia sp., including salpingitis, urethritis, cervicitis and trachoma; treatment or prophylaxis of respiratory diseases; treatment or prophylaxis of diseases caused by Bordetella sp., including whooping cough; treatment or prophylaxis of diseases caused by respiratory syncytial virus, including lower respiratory disease.

Also disclosed is a method for the manufacture of the Immunogenic Complex of the invention comprising admixing a Ribosomal Complex with a Polynucleotide Molecule, wherein the Ribosomal complex is from a Microbe and the Polynucleotide Molecule is from, derived from or deduced from a Microbe or a virus. Preferably the Ribosomal complex and the Polynucleotide Molecule are incorporated in a polymeric matrix consisting essentially of chitosan-EDTA Bowman-Birk Inhibitor conjugate. In other aspects, the Ribosomal complex and the Polynucleotide Molecule are preferably incorporated in microparticles essentially composed of carboxymethylethylcellulose-coated poly[dl-lactide-coglycolide]. In further aspects of the method for the manufacture of the Immunogenic Complex according to the invention, the Ribosomal complex and the Polynucleotide Molecule are non-covalently coupled to each other, whereby the Ribosomal Complex is covalently conjugated to poly (L-lysine) and the Polynucleotide Molecule is subsequently condensed onto said Ribosomal Complex-polycation conjugate by ionic interaction. In yet further aspects of the method for the manufacture of the Immunogenic Complex according to the invention, the Ribosomal complex and the Polynucleotide Molecule are covalently coupled to each other by treatment of the Ribosomal Complex with 2-iminothiolate, followed by addition of Polynucleotide Molecule and mild ultraviolet irradiation.

In another aspect, the Ribosomal complex and the Polynucleotide Molecule are covalently coupled to each other by reduction of 5′ thio-derivatized Polynucleotide Molecule, via the freed thiol-group, to maleimide-derivatized Ribosomal Complex. Also encompassed is a method for the manufacture of the pharmaceutical composition of the invention comprising admixing the Immunogenic Complex of the invention with a pharmaceutically acceptable carrier, diluent or other excipient.

The invention also provides methods of administration of the Immunogenic Complex or the pharmaceutical composition according to the invention to humans and/or animals. Examples include oral administration of the Immunogenic Complex or the pharmaceutical composition upon suspension in a drinkable liquid, Topical administration of the Immunogenic Complex or the pharmaceutical composition contained in a liquid, a gel or cream and applied to epithelial cell surfaces, in particular to surfaces of infected or infection-prone areas, nasal administration of the Immunogenic Complex or the pharmaceutical composition contained in a liquid aerosol or droplet dispenser, by inhalation upon containment in a peroral liquid or dry powder aerosol, and Rectal or vaginal or uteral application of the Immunogenic Complex or the pharmaceutical composition contained in a suppository or as a gel or cream.

### DETAILED DESCRIPTION OF THE INVENTION

The present invention pertains to Immunogenic Complexes, the production and formulation thereof, methods of application and the use of Immunogenic Complexes as either prophylactic vaccines or therapeutic agents in pharmaceutical compositions. It will be readily apparent to one skilled in the art that various substitutions and modifications may be made to the invention disclosed herein without departing from the scope and spirit of the invention. In accordance with the present invention there may be employed conventional molecular biology, microbiology, and recombinant DNA techniques within the skill of the art. Such techniques are fully explained in the literature.

According to the present invention an appropriately produced Immunogenic Complex, comprising Ribosomal Complex and Polynucleotide Molecule, jointly administered to an individual's cells, preferably cells of the Mucosal Immune System, elicits a strong and broad immune reaction against antigens present on the Ribosomal Complex and against the Antigen encoded on the Polynucleotide Molecule, once the Polynucleotide Molecule is present in the host's cells. The resulting antigen-binding molecules of the hereby vaccinated host can recognise, bind and direct the destruction of microbe and/or virus from which the antigens originated, were derived or were deduced.

Thus the Immunogenic Complex provided by present invention is particularly useful to protect against and combat microbial and viral pathogens.

Therefore, if appearing herein, the following terms shall have the definitions set out below.

As used herein, the term “Microbes” refers to bacteria, protozoa and fungi.

As used herein, the term “Ribosomal Complex” refers to a complex which is essentially composed of the subunits of ribosomes (50 S and 30 S subunits in bacteria and 60 S and 40 S subunits in eucaryotes) which carry on their surface minor fractions of the microbial cellular membrane or cell wall components. An important feature of the invention is that the ribosomal subunits in the Ribosomal Complex retain sufficient integrity to preserve substantially the double-stranded nature of the large r-RNA's (16 S and 23 S in bacteria; 18S and 28S in eukaryotic cytosol) contained in the ribosomal subunits. Preferably, the Ribosomal Complex is largely particulate in nature, preferably having a granular (versus soluble) structure.

As used herein, the term “Antigen” refers to any protein, or polypeptide derived, deduced or part of such Antigen, that is able to interact specifically with an Antigen recognition molecule of the immune system, such as an antibody (immunoglobulin) or T cell-antigen receptor. An antigenic portion of a molecule can be that portion that is immuno-dominant for antibody or T cell receptor recognition, or it can be a portion of such protein which when fused to a carrier molecule for immunisation is capable of inducing specific Antigen recognition molecules that will bind to it. A molecule that is antigenic need not be itself immunogenic, i.e., capable of eliciting an immune response without a carrier molecule.

As used herein, the term “Polynucleotide Molecule” refers to DNA or RNA which comprise a nucleotide sequence that encodes an antigen protein or encodes a polypeptide that is part of, derived or deduced from said antigen protein. Polynucleotide Molecule may consist genomic DNA, cDNA, synthesised DNA or a combination thereof or an RNA molecule such a Positive RNA stranded viral genome or part thereof or mRNA.

As used herein, the term “nucleotide sequence” can refer to both DNA and RNA molecules.

As used herein, the term “transcription unit” refers to the sequences of a gene, that can be transcribed from DNA to mRNA, as well as refer to nucleotide sequences on mRNA.

A “DNA transcription unit” is DNA nucleotide sequence, bounded by an initiation site and termination site, that is transcribed to produce a primary transcript. As used herein, a “DNA transcription unit” includes at least two components: protein-encoding DNA and transcriptional promoter element or elements. Protein-encoding DNA can encode a single antigen or multiple antigens, such as antigens from two or more different proteins of infectious agents. A DNA transcription unit can optionally include additional sequences, such as: enhancer elements, splicing signals, termination and polyadenylation signals, viral replicons and bacterial plasmid sequences. In the present invention, a single type of DNA transcription unit can be administered, or a combination of two or more types of DNA transcription units can be administered.

The DNA transcription unit can be produced by a number of known methods. For example, DNA encoding a selected antigen can be inserted into expression vectors know to the man skilled in the art and available through many suppliers.

A “vector” is a genetically engineered replicon, such as plasmid, phage or cosmid, to which a heterologous DNA segment is attached so as to bring about the replication of the attached segment. A “replicon” is any genetic element (e.g., plasmid, chromosome, virus) that functions as an autonomous unit of DNA replication in vivo, i.e., capable of replication under its own control. A vector can have inserted a DNA transcription unit such that the transcription unit can be multiplied by vector replication and in certain cases or circumstances allow expression of the gene on the DNA transcription unit.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, viral sequences, and synthetic DNA sequences. Since the coding sequence is intended for expression in a eukaryotic cell, a polyadenylation signal and transcription termination sequence will usually be located 3′ to the coding sequence.

A “promoter” is a DNA regulatory region capable of binding RNA polymerase in a cell and initiating transcription of a downstream (3′ direction) coding sequence. For purposes of defining the present invention, the promoter is bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of RNA polymerase.

In specific embodiments of this invention the expression of Antigen can be controlled by any of a number of promoter/enhancer elements known in the art, but these regulatory elements must be functional in the host selected for expression. Promoters which may be used to control Antigen gene expression and are cloned 5′ of the Antigen coding sequences on the Polynucleotide Molecule include, but are not limited to retrovirus promoters and mammalian promoters. More specific examples that can be used to practice present invention, especially in the production of Immunogenic Complex or Bacterio-viral Immunogenic Complex or Heterologous Immunogenic Complex to be used in a vaccine for humans, include cytomegalovirus (CMV) immediate early promoter, the chicken beta-actin promoter, mouse mammary tumor virus (MMTV) promoter, human immunodeficiency virus long terminal repeat (HIV LTR) promoter, human actin promoter, human myosin promoter, the SV40 early promoter region (Benoist and Chambon, Nature, 290:304-310 [1981]), the promoter contained in the 3′ long terminal repeat of Rous sarcoma virus (Yamamoto, et al., Cell, 22:787-797 (1980]), and the herpes thymidine kinase promoter (Wagner et al., Proc. Natl. Acad. Sci. U.S.A., 78:1441-1445[1981]).

In a preferred embodiment, the human immunoglobulin gene control region (Alexander et al., 1987, Mol. Cell. Biol. 7: 1436-1444) and/or the mouse mammary tumor virus control region (Leder et al., 1986, Cell 45: 485495) are used as they are active in lymphoid cells.

Examples of polyadenylation signals useful to practise the present invention, especially to practise the invention in vaccines for humans, include but are not limited to HIV LTR polyadenylation signal, the SV40 polyadenylation signal, the short (117 bases) bovine growth hormone (BGH) gene transcriptional termination sequence and the rabbit β-globin gene transcriptional terminator sequence.

Examples of enhancer elements useful to practise the present invention, especially to practise the invention in vaccines for humans may be selected from the group including but not limited to enhancer sequences identified old transcription units encoding human actin, human myosin, human hemoglobin, human muscle creatine, CMV, RSV and EBV.

A coding sequence is “under the control” of transcriptional and translational control sequences in a cell when RNA polymerase transcribes the coding sequence into mRNA, which is translated into the protein encoded by the coding sequence.

A “signal sequence” is included at the beginning of the coding sequence of a protein to be expressed on the surface of a cell. This sequence encodes a signal peptide, N-terminal to the mature polypeptide, that directs the host cell to translocate the polypeptide. Translocation signal sequences can be found associated with a variety of proteins native to eukaryotes and prokaryotes, and are often functional in both types of organisms.

As used herein, the term “Adhesin” refers to any protein embedded in or otherwise associated with the surface of Microbes that is involved in the attachment to host cells such as absorptive enterocytes, M-cells, dendritic cells, macrophages, erythrocytes, fibroblasts and epithelial cells or in binding to components of the extra-cellular matrix that embeds host cells such as fibronectin, laminin, collagen, fibrogen, vitronectin, heparin sulphate. “Adhesin” also includes any polypeptide derived from or corresponding to part of such protein that, under appropriate conditions, can still induce an immune response against said Adhesin. “Adhesin” also includes the protein complexes of colonisation factor antigens such as those present in bacterial fimbriae and fungal hyphae.

The phrase “pharmaceutically acceptable” refers to molecular entities and compositions that are physiologically tolerable and do not typically produce an allergic or similar undesirable reaction, such as gastric upset, dizziness, fever and the like, when administrated to a human. Preferably, as used herein, the term “pharmaceutically acceptable” means fulfilling the guidelines and approval criteria of a European Community country's Drug Registration Agency concerning products to be used as a drug, or means that the pharmaceutically acceptable compound, composition, method or use, is listed in the European Community country's Pharmacopoeia or other generally recognised pharmacopoeia for use in animals, and more particularly in humans.

The term “pharmaceutical carrier” refers to a diluent, adjuvant, excipient, or vehicle with which the compound is administered. Such pharmaceutical carriers include but are not limited to sterile liquids, such as water and oils, including those of petroleum, oil of animal-, vegetable-, or synthetic origin, such as whale oil, sesame oil, soybean oil, mineral oil and the like. Water or aqueous solutions, saline solutions, and aqueous dextrose and glycerol solutions are preferably employed as carriers, particularly for injectable solutions, droplet-dispensed solutions and aerosols.

The term “adjuvant” refers to a compound or mixture that enhances the immune response to an antigen. An adjuvant can serve as a tissue depot that slowly releases the antigen and also as a lymphoid system activator that non-specifically enhances the immune response Preferably, the adjuvant is pharmaceutically acceptable.

As used herein, the term “Immunogenic Complex” refers to a complex comprising at least following elements: Ribosomal Complex and Polynucleotide Molecule encoding Antigen of a Microbe.

An Immunogenic Complex can contain Ribosomal Complex and Polynucleotide Molecules of several species of Microbes. This is particularly advantageous for disease prevention and/or treatment of diseases, which may be caused or aggravated by multiple pathogens (e.g. periodontal disease, Common Cold, Broncheolitis, otitis, etc.).

As used herein, the term “Heterologous Immunogenic Complex” refers to an Immunogenic Complex comprising Ribosomal Complex and Polynucleotide Molecule, that originate from different, or multiple Microbes, whereby from one or more species of Microbes, only Ribosomal Complex but not Polynucleotide Molecule or only Polynucleotide Molecule and not Ribosomal Complex, is included.

This is particularly advantageous in cases where an immune response is desirable against a given pathogen in a complex of Microbes against which one wishes to use the Heterologous Immunogenic Complex but for which pathogen the use of both Ribosomal Complex and Polynucleotide Molecule is not desirable. In such cases it is preferable to include only Ribosomal Complex or only Polynucleotide Molecule of this Microbe. Concerning Polynucleotide Molecule, this is the case when, for example, appropriate antigen encoding genes of a Microbe are not, or are poorly characterised (e.g. Campylobacter rectus) or are difficult or expensive to identify, characterise and isolate or in cases that antigenicity requires a certain composition e.g.hetero-multimerisation that cannot simply be obtained by the sole expression of the antigen gene in the host cell. Examples where it may be desirable not to include the Ribosomal Complex of one or more Microbes in a Heterologous Immunogenic Complex, are cases where one or more of said Microbes are difficult or expensive to produce in large quantities (e.g. many oral treponemes associated with periodontal problems); another obvious reason to eliminate for a target species the Ribosomal Complex, is where an immune response is induced which cross-reacts with host tissue (unpublished observation with Streptococcus pyogenes A).

As used herein, the term “Bacterio-viral Immunogenic Complex” refers to an Immunogenic Complex, containing at least following elements: Ribosomal Complex of bacteria and Polynucleotide Molecule encoding Antigen of virus.

Similarly to Immunogenic Complex, the Bacterio-viral Immunogenic Complex is advantageous for disease prevention and/or treatment of diseases resulting from infection by several pathogens. In particular are concerned diseases which may initiate as result of viral infection which facilitate colonisation of bacterial pathogens, that super-infect and aggravate and prolong the disease (e.g. Common Cold, Broncheolitis, diarrhoea's, Meningitis caused by Neisseria meningitis following infection by respiratory syncytial virus, etc.).

In one aspect, the present invention concerns the preparation and use of Ribosomal Complex in Immunogenic Complex. As included therein, the Ribosomal Complex is a surprisingly good ingredient as it appears to impart antigenic activity against the Microbe from which it is extracted. The Ribosomal Complex also functions as a vehicle for delivery of Antigens to the Mucosal Immune System, in particular Polynucleotide Molecules which encode Antigen of same or different Microbe or of virus, and functions as an adjuvant by enhancement of non-specific immune response.

Protocols describing the preparation of Ribosomal Complex (RC) from Microbes are available in the literature and can be adapted where needed by those skilled in the art. For example, the preparation of RC from bacteria can be done essentially as described by Youmans and Youmans, 1965, with following adaptations: The bacterial culture is grown in regular broth at a temperature and atmospheric conditions optimal for the species. Subsequently the cells, whilst still in log phase growth, are rapidly cooled to 10° C., harvested by low-speed centrifugation (10.000×g for 10 min.), washed three times in a phosphate buffer (0.01M, pH 7.0) containing 0.01 M MgCl₂ (PMB) and frozen at −80° C. In general, but particularly when using virulent Microbes (pathogenic), is recommended to kill the cells prior to further use for example by treatment with formalin as described by Michalek and McGhee, 1977, and adjust concentrations to 10⁸ bacterial or fungal cells/ml or 10⁷ protozoa/ml. The preparation can be established to be sterile when no multiplication occurs upon inoculation on Sheep blood and Mitis Salivarius agars (DIFCO) or other adapted rich culture medium. Aliquots are stored at −80° C. Subsequently they are thawed rapidly at 37° C., and 1 g of whole cells is re-suspended with 1 g of microglass beads (0.17-0.18 mm) in 1 ml of PMB to which 3 μg/ml Dnase (SIGMA) is added. Shaking for three 2-min. cycles in a Braun homogenizer disrupts the cells. Intact cells and debris are removed by two centrifugations (27.000×g+47.000×g, 10 min.). Preparation of ribosomes from fungi and protozoa follow essentially the same procedure but require adaptation of culture conditions and lysis methods. Given that culture conditions of pathogenic Microbes that can be cultivated are widely available in published literature, preparation of ribosomes from such Microbes is well within the capacity of a person skilled in the art.

Integrity of the ribosomal subunits is important. In particular the stabilisation of enclosed large ribosomal RNA's by divalent cations such as provided by MgCl₂, concentration that may need adaptation depending on the Microbe and extraction protocol, method that the man skilled in the art shall know to adapt. The ribosomes in the supernatant can be harvested by centrifugation at 180.000 to 250.000×g for 2 to 3 hr and then subjected to 5 successive washes in PMB at 180.000 to 250.000×g for 2 to 3 hr each. The ribosomal preparation is then clarified twice by two 20-min. centrifugations at 47.000×g and the supernatant is filtered through a sterile 0.45 μm Millipore filter (Millipore Filter Corp.). Non-dissociated (=intact) ribosomes can be prepared from gram-negative, Rnase-minus mutant bacteria such as Escherichia coli MRE600 following the method of Staehilin et. al., 1969, with modifications as described by M. M. Yusupov and A. S. Spirin. 1988. The preparation can then adjusted to, for example, 20 mg/ml on the basis of protein content by standard protein quantification methods and is subsequently maintained at −80° C. until used. Characterisation of the ribosomal fraction and purity can be determined by spectral analysis at 235, 280 and 260 nm in order to determine the contamination of ribosomal RNA by DNA. Polyacrylamide gel electrophoresis permits to evaluate the presence of ribosomal proteins and potential contaminating proteins. The degree of intactness can be evaluated by loading a sample of the original homogenate onto a 10% to 40% sucrose gradient, containing an appropriate concentration of Mg Cl₂ and centrifugation. The elusion profile of the sucrose gradient will show the different peaks: 100S=dimers of 70S ribosomes, 70S=intact ribosomes, 60S=interacting 50S and 30S ribosomal subunits, 50S=large ribosomal subunit, 30S=small ribosomal subunit, material less than 30 S=degradation products and contaminants. In good preparations that target non-dissociated ribosomes, the 70S peak contains over 80% of all material. Optionally, the 70S peak containing the target non-dissociated ribosomes may constitute at least 50%, 60%, 70% or 90% of all material.

The term “isolated” requires that the material be removed from its original environment (e. g., the natural environment if it is naturally occurring). For example, a naturally-occurring polynucleotide, polypeptide, Ribosomal Complex or ribosomal subunit present in a living animal is not isolated, but the same polynucleotide or DNA or polypeptide, separated from some or all of the coexisting materials in the natural system, is isolated. Such polynucleotide could be part of a vector and/or such polynucleotide, polypeptide, Ribosomal Complex or ribosomal subunit could be part of a composition, and still be isolated in that the vector or composition is not part of its natural environment.

The term “purified” does not require absolute purity; rather, it is intended as a relative definition. Purification of starting material or natural material (e.g. polynucleotides, polypeptides, ribosomal subunits or Ribosomal Complex) to at least one order of magnitude, preferably two or three orders, and more preferably four or five orders of magnitude is expressly contemplated. As an example, purification from 0.1% concentration to 10% concentration is two orders of magnitude. The term “purified” is further used herein to describe a polynucleotides, polypeptides, ribosomal subunits or Ribosomal Complex which has been separated from other compounds including, but not limited to, polypeptides or polynucleotides, carbohydrates, lipids, etc. The term “purified” may be used to specify the separation of monomeric polypeptides of the invention from oligomeric forms such as homo- or hetero-dimers, trimers, etc. A polynucleotide is substantially pure when at least about 50%, preferably 60 to 75% of a sample exhibits a single polynucleotide sequence and conformation. A substantially pure polypeptide, polynucleotide, ribosomal subunit or Ribosomal Complex typically comprises about 50%, preferably 60 to 90% weight/weight of a polypeptide, polynucleotide, ribosomal subunit or ribosomal RNA sample, respectively, more usually about 95%, and preferably is over about 99% pure. Polypeptide and polynucleotide purity, or homogeneity, is indicated by a number of means well known in the art, such as agarose or polyacrylamide gel electrophoresis of a sample, followed by visualizing a single band upon staining the gel. For certain purposes higher resolution can be provided by using HPLC or other means well known in the art.

Another aspect of the present invention concerns the preparation and use in Immunogenic Complex of Polynucleotide Molecule. As included therein, Polynucleotide Molecule is a surprisingly good ingredient, targeted to cells of the immune system by the Ribosomal Complex, and whereby the Antigen encoded on Polynucleotide Molecule appears to be expressed in immuno-competent host cells.

In a preferred embodiment, Polynucleotide Molecule is a DNA molecule that comprises a DNA transcription unit that encodes an Antigen. Upon transcription/translation of the Antigen-encoding DNA transcription unit in the host cell, Antigen is produced in the host cell. This can induce an immune response in the host leading to production of Antigen recognition molecules (e.g. antibodies) which allows the host to detect, bind and destroy microbes and/or viruses carrying said Antigen or related Antigens that are still recognised by the Antigen recognition molecules.

The genes of many Antigens of Microbes and viruses have been identified, cloned and are described in the literature or available in public databases. Their coding sequences are known and those skilled in the art know how to use this information to reclone, synthesise or sub-clone the transcription unit carrying the gene into an appropriate plasmid vector, linked with regulatory sequences which assure the expression of the gene once introduced in the host cell.

Although it is possible to induce strong and specific humoral responses by immunisation with Polynucleotide Molecules encoding secretory proteins, whether coding sequences of the Antigen include secretory signal sequences or not, for certain purposes it may be desirable to engineer recombinant antigen genes containing signal sequences in frame with the mature protein encoding sequences. This is particularly desirable if the conformational state and/or post-translational modifications of the mature protein is/are important for the induction of an effective immune response. An example of signal sequences useful to practice the present invention and in particular to practise the present invention for use in vaccines for humans, is the human tissue plasminogen activator (tPA) endoplasmatic reticulum-targeting signal sequence. This tPA signal sequence is fused in frame at the 5′end of an Antigen-encoding polypeptide sequence on the Polynucleotide Molecule.

In preferred embodiments of the present invention, preferred Antigens of Microbes are Adhesins. The effect of using in the Immunogenic Complex selected Polynucleotide Molecules which carry DNA transcription units encoding Adhesin, is the enhancement of the immune response specifically against said Adhesins and further contributes to effective immune exclusion of the target Microbe.

Adhesins and their genes have been widely studied for many Microbes and protocols describing the cloning of Adhesin genes in expression vectors, are available in the literature and can be adapted where needed by those skilled in the art. A non-exhaustive list of examples of microbial Adhesin genes that can be used to practise the present invention, by the appropriate integration of polynucleotide sequence or sequences derived or deduced from said Adhesin gene, in the Polynucleotide Molecule of Immunogenic Complex are: The products of Staphylococcus aureus genes fnbA and fnbB, encoding 110 and 98 kDa proteins respectively; the gene encoding porin OmpC protein of Salmonella typhimurium; the DNA transcription unit with coding sequences for polypeptide segments PAK 128-144, PAO 128-144, corresponding respectively to amino acid sequences of the C-terminal receptor binding regions of strains PAK & PAO of Pseudomonas aeruginosa pilin protein; the spaP gene encoding full length Streptococcus mutans non-fimbrial cell surface antigen SA I/II; transcription unit carrying the coding sequences for the polypeptide derived from SA I/II, that spans the residues 1025-1044 in the C-terminal domain.; The wil gene encoding an adhesin (WI-1)of Ajellomyces dermatitidis yeast; the hwp 1 gene encoding Hyphal Wall protein, an adhesin of Candida albicans, expressed during the hyphal phase of the pathogen; surface Adhesin gene fab1 of Streptococcus parasanguis; Treponema denticola major outer sheat protein gene msp; Porphyromonas gingivalis fimbrilin gene fimA; the fhaB gene encoding filamentous hemagglutinin of Bordetella pertussis; the hap gene of Haemophilus influenzae, ubiquitously present among different strains of H. influenzae; the PsaA gene encoding pneumococcal surface adhesin A of Streptococcus pneumoniae, expressed by 90 serotypes of S. pneumoniae; the PrtF gene of Streptococcus pyogenes, encoding the fibronectin binding protein F; the gene encoding the Colonisation Factor Antigen CFA/IV of entero-toxigenic Escherichia coli; the clpG gene which encodes the capsule-like surface antigen CS31A harboured by bovine and human entero-toxigenic or septicemic Escherichia coli and Klebsiella pneumoniae strains responsible for nocosomial infections; the HBHA gene encoding heparin-binding hemagglutinin in Mycobacterium tuberculosis. Polynucleotide sequences, referred herein as examples for use in Immunogenic Complex, are disclosed in the GenBank database and are listed, under the accession number provided by the USA National Center for Biotechnology Information, in Table 1. The NCBI web site with link to the database is also provided.

Other microbial surface proteins may also be good Antigens. As for Adhesin encoding genes, the antigenicity of selected surface proteins requires empirical testing by including the appropriate polynucleotide sequences of such Antigen encoding genes or the polynucleotide sequences derived or deduced from said Antigen encoding genes, in the Polynucleotide Molecule as described in this invention, and subsequent evaluation of antigen-specific immune induction in experimental animals such as mice, rats, pigs, primates, etc. A few non-limiting examples of genes encoding such secreted proteins which can be used to practise present invention by their proper inclusion on the Polynucleotide Molecule of Immunogenic Complex or Heterologous Immunogenic Complex are: the pneumococcal surface protein gene, PspA of Streptococcus pneumoniae; the surface antigen D15 of Haemophilus influenzae, type B. In a preferred embodiment, the mutant toxA gene contains at least two mutations, preferable in the third domain which abrogates enzymatic activity of the toxin (Wozniak, et al., Appl. environ. Microbiol. 61, 1739-1744); the immuno-derterminant antigen P39 and P35 genes of Borrelia burgdorferi, etiologic agent of lyme disease; gene encoding Chlamydia pneumoniae 53 Kda antigen peptide; gene for Chlamydia trachomatis 59 kDa immunogenic protein SK59. Polynucleotide sequences, referred herein as examples for use in Immunogenic Complex, are disclosed in the GenBank database and are listed, under the accession number provided by the USA National Center for Biotechnology Information, in Table 1. The NCBI web site with link to the database is also provided.

In other embodiments the Polynucleotide Molecule of Bacterio-viral Immunogenic Complex can carry transcription units or the polynuceotide sequences derived or deduced from many viral Antigen genes of which a non exhaustive list follows: Gene for the fusion (F) protein of respiratory syncytial virus (RSV); the gene encoding the attachment G glycoprotein of RSV; the polypeptide encoding sequence for the junction of the glycoprotein G with the fusion protein F of RSV; the polynucleotide sequence for the central conserved domain of the G glycoprotein of (RSV) which spans the amino acids (N-terminal to C-terminal direction) 124 to 230; the gene encoding hemagglutinin (HA) of influenza virus; the gene encoding neuraminidase (NA) of influenza virus; the gene encoding the nucleoprotein (NP) of influenza virus; the genes encoding AgD, SgD or CgD antigens of Bovine herpesvirus-1; gene encoding glycoprotein B (gB) or glycoprotein D (gD); gene encoding the VP4 antigen of Group A rotavirus; gene encoding HA or nucleoprotein (NP) of measles virus; gene encoding the S protein of Hepatitis B virus (HBV); gene encoding the core protein, (HbcAg), or HbeAg or HbsAg of HBV; genes encoding HCV proteins such as core, S, E1 and E2; the gene encoding glycoprotein gp 160 or envelope protein or the gag/pol gene or the rev gene or tat gene or nef gene of human immunodeficiency virus (HIV); the gene encoding the nucleoprotein of the lymphocytic choriomeningitis virus; the gene encoding the major capsid protein L1 of papillomavirus ; the gene encoding glycoprotein of rabies virus; the gene encoding envelope protein or Vp4 or Vp6 or Vp7 of rotavirus; the gene encoding the viral envelope (E) protein, the gene encoding the precursor for membrane (prM) protein and the gene encoding the non-structural protein NS1 of Murray Valley encephalitis virus (MVEV); the genes encoding prM or E proteins of Japanese encephalitis virus. Polynucleotide sequences, referred herein as examples for use in Immunogenic Complex, are disclosed in the GenBank database and are listed, under the accession number provided by the USA National Center for Biotechnology Information, in Table 1. The NCBI web site with link to the database is also provided.

Protocols describing the preparation of vectors and vectors containing selected Antigen-encoding transcription units are available in the literature and can be adapted where needed by those skilled in the art. Many types of vectors can be used for production of Polynucleotide Molecule, to be used in Immunogenic Complex. Such vectors include but are not limiting to: plasmid vectors, cosmid DNA vectors, bacterial artificial chromosomes (BAC), bacteriophage vectors (e.g. Lambda), yeast vectors, human or animal viruses such as adenovirus, alphavirus-based vectors or vaccinia virus-based vectors. As mentioned earlier is well within the abilities of those skilled in the art to properly clone genes encoding antigens into above-mentioned different types of vectors.

In a specific embodiment of this invention the vector consists of a DNA plasmid based on pUC19 (New England Biolabs Inc., USA). It consists of the ColE1 multi-copy Escherichia coli plasmid origin of replication and the β-lactamase gene (la) conferring resistance to ampicillin for maintenance and production in E. coli. The lac operon is removed from pUC19 by a partial digestion with Hae II restriction enzyme and gel purification. The appropriate Hae II fragment (site at bp 1050 not cleaved) is subsequently blunted by T4 DNA polymerase treatment and is dephosphorylated with calf intestinal alkaline phosphatase. As expression cassette, the human immunoglobulin gene control region (as promoter), a poly-linker cloning site (carrying sites for EcoR I, Apo I, Ban II, Ecll36 II, Sac I, Kpn I, Acc65 I, Ava I, Xma I, Sma I, BamH I, Xba I, Sal I, Hinc III, Acc I, BspM I, Sph I and Hind III) followed by the rabbit β-globin transcriptional termination sequences, consisting essentially of polyadenylation signal and downstream termination elements, is used. This fragment is also blunted and can be ligated into the above described linearised vector. The resulting circular plasmid is here called pBen0161. The target Antigen gene can be cloned in the poly-linker cloning site of the above described plasmid vector. Table 2 lists the components of pBen0161 by providing the NCBI accession number and source organism of DNA plasmid pUC 19, the promoter of human immunoglobulin Heavy Chain and the gene plus 3′flanking region of rabbit β-globin. The NCBI web site with link to the database is also provided.

In a preferred embodiment, plasmid DNA containing a DNA transcription unit for an Antigen gene is prepared from Escherichia coli for use as Polynucleotide Molecule in Immunogenic Complex or Bacterio-viral Immunogenic Complex or Heterologous Immunogenic Complex. Plasmid DNA preparation is well established in the art; the skilled person will know to adapt protocols depending on the strain used. For example, an E. coli strain, preferentially strain DH5α, containing the expression vector, preferentially the pUC19 derivative, pBen0161, containing a DNA transcription unit for the Antigen gene, is taken from an aliquoted sample of the referenced stock stored in glycerol and is freshly grown on solid broth containing 100 μg/ml ampiciline. Individual colonies are picked and used to inoculate a solution of 500-ml Terrific Broth per 1-liter shake flask. Growth is permitted overnight at 37° C. with vigorous aeration. Cells are harvested at end-log phase as determined by OD₆₀₀ measurement (after empirical determination of OD₆₀₀ value corresponding to end-log phase) and lysed, for example by a modification of the alkaline, NaDodSo₄ procedure. The modification consists of increasing the volumes three-fold for cell lysis and DNA extraction. DNA is purified by double banding on Cesium-Chloridel-Ethidium-Bromide (CsCl-EtBr) gradients; the EtBr is removed by 1-butanol extraction. The resulting DNA is phenol/chloroform-extracted and Ethanol-precipitated. DNA is resuspended in appropriate solvent for coupling to Ribosomal Complex as described below. [10 mM Tris, 1 mM EDTA (TE) buffer, pH 8 for transfections and in 0.9% NaCl for injection]. The concentration and purity of each DNA preparation is determined in an aliquot by OD_{260/280} ratios and are superior to 1.8

The optimal ratio of Ribosomal Complex to Polynucleotide Molecule in the Immunogenic Complex, Heterologous Immunogenic Complex and in the Bacterio-viral Immunogenic Complex depends on several factors including the method used to couple both components, the method and tissue to which the complex is delivered (targeting efficiency of Ribosomal Complex), the size of the Polynucleotide Molecule and the level and type of specific humoral and cellular immune induction observed as result of expression of the Antigen gene encoded on the Polynucleotide Molecule. Consequently, the optimal ratio of the respective components is best determined empirically. This can be done by preparing Immunogenic Complex or Heterologous Immunogenic Complex or Bacterio-viral Immunogenic Complex with different ratio's of the components ranging preferentially from 0.05 to 20 [weight/weight] and evaluating, for different dosed Immunogenic Complexes, the expression levels of the antigen gene in vitro, in transfected cells (of a type known to express genes under the chosen promoter and preferably from the species which is targeted for immunisation) and i,i vivo, using appropriate animal models (e.g. mice, rats, rabbits, pigs, monkeys). Hereby specific antibody titers against the Antigen are compared, preferentially using monoclonal antibodies and ELISA readings are compared following the interaction of serum or other bodily fluid samples (containing polyclonal antibodies induced in selected animals against the relevant target Microbe and/or virus) with appropriately diluted samples of said target Microbe and/or virus.

It is an object of the invention to ensure joint delivery of the Immunogenic Complex to the Mucosal Immune System of vertebrate animals including humans by different methods depending on the target tissue & host, delivery method and acceptable production complexity.

In a specific embodiment of this invention, Ribosomal Complex and Polynucleotide Molecule are co-delivered by joint encapsulation in a micro-particle or joint complexion in a protective matrix. These embodiments are preferred when using present invention for delivery of Immunogenic Complex as oral vaccine to the gastrointestinal tract (GIS) in order to protect it from the low pH environment and excessive degradation by enzymes such as pepsins, trypsin, chymotrypsin, elastase and carboxypeptidase. The use of such polymeric substances as protective carriers is well documented. The skilled man will know to select and adapt protocols such as to guarantee that the encapsulated or matrix-embedded Immunogenic Complex (IC) has preserved, to a substantial extent, the integrity of the ribosomal subunits of the Ribosomal Complex (RC). A preferred polymeric substance used to produce micro-particle carrier is the hydrophobic polymer carboxymethylethylcellulose (CMEC) coated poly[dl-lactide-coglycolide] (PLG). Immunogenic Complex (IC) contained in CMEC coated PLG particles can be prepared as follows: An aqueous solution of IC (40 ml, 20 mg/ml) is emulsified with 200 ml of a 4% solution of PLG copolymer (Resomer RG 503, Mw 34,000) in dichloromethane (DCM) using a Silverson homogenizer for 3 min. at 10,000 rpm to produce the primary emulsion. The resulting w/o emulsion is then re-emulsified for 10 min. at high speed with a solution of CMEC to produce a double emulsion (w/o/w). Different concentrations of CMEC are best tested (2.5%-8%), adding 0.2 M NaOH to yield a final pH of approximately 6. The w/o/w emulsion is stirred magnetically for 12 h at room temperature and under reduced atmospheric pressure to allow solvent evaporation. The micro-spheres are isolated by centrifugation, washed 3 times in double distilled water and lyophilised. The product can be stored in a desiccator at a temperature of −18 Celsius. Particles can be sized by laser difractometry using a Malvern 2600D laser sizer. Particle size is expressed as volume mean diameter. Encapsulation (10%-50% efficiency) can be achieved in enteric-coated PLG micro-particles with volume average diameter of less than 8 μm.

Another preferred substance for delivery to the GIT consists of the polymeric matrix chitosan-EDTA Bowman-Birk inhibitor conjugate (CEBBI). IC complexed in CEBBI tablets for oral delivery can be obtained as follows: 18.15 g of EDTA (ethylene-diamine-tetra-acetic acid; Sigma, St.Louis, Mo.) are dissolved in 100 ml of demineralised water and the pH-value adjusted to 6.0 with 5 N NaOH. To this solution 100 ml of an aqueous solution of 1% (w/v) chitosan HCL pH 6.0 (poly-[1-4]-β-D-glucosamine; Sigma, St-Louis, Mo.) and 5 ml of an aqueous solution of 2.27 g of EDAC (1-ethyl-3-(dimethylamionpropyl) carbo-diimide hydrochloride; Sigma, St-Louis, Mo.) are added. The reaction mixture is incubated at room temperature under permanent stirring for 12 h. The resulting conjugate is isolated by exhaustively dialysing against demineralised water, 50 mM NaOH and once more against demineralised water. The purified product is precipitated by pouring the dialysed polymer solution rapidly into an unstirred bath of non-solvent (acetone) at solvent to non-solvent ratio of 1:200, washed in acetone, and air-dried. The dried polymer can be stored at room temperature until use. 120 mg of this polymer are dissolved in 20 ml of demineralised water. EDAC and SNHS (sulfo-N-hydroxy-succinimide; Pierce, Oud-Beijerland, Nl) are added in a final concentration of 0.1 M and 5 mM, respectively, and the reaction mixture is incubated for 60 min. at room temperature under permanent stirring. Thereafter, 12 mg of previously demineralised (PD10 column; Pharmacia, Uppsala, Sweden) Bowvman-Birk Inhibitor (BBI) is added, and the reaction allowed to proceed for 12 h. The reaction mixture is dialysed for 6 h against demineralised water and then centrifuged for 30 min at 17.000 g (Sorvall RC5C, Dupont). The supernatant, containing the unbound inhibitor and coupling reagent, is discarded. The remaining pellet of the polymer-BBI conjugate is diluted with an at least 10-fold amount of demineralised water, centrifuged and the supernatant removed again. This purification step is repeated 10 times. The isolated polymer-BBI conjugate is precipitated in acetone as described above and stored at −20° C. until use. Lyophilised IC is added at 1% (w/w) to chitosan-EDTA (55%), chitosan-EDTA BBI conjugate (14%/o) and D-mannitol (30%), homogenised in a mortar and pressed (Hanseaten, Hamburg, Germany) to 250 mg pellets. As controls one can use tablets prepared identically but with following difference: no IC added and Chitosan-EDTA concentration at 56%.

The amount of polyreric substance versus Immunogenic Complex employed in such vaccines will vary depending upon the exact Pharmaceutical Carrier used. Adjustment and manipulation of established dosage ranges used with traditional carrier molecules for adaptation to the present invention is well within the ability of those skilled in the art, however it is preferred to keep the size of the micro-particles between 0.1 and 8 μm in diameter.

If no excessive degradation of the Immunogenic Complex (IC) is expected by enzymes of the targeted mucosa, it is preferred to deliver the IC without said polymeric substances, thus avoiding additional preparation steps, product recovery losses and possible contra-indications originating from side-products, intermediates, remaining chemical impurities or the carrier itself. However, in such cases it is preferred to couple the Ribosomal Complex (RC) and Polynucleotide Molecule (PNM) to assure joint delivery to the targeted mucosal surface. The RC and PNM can be coupled via several methods such as complexing PNM to RC-polycation conjugates or linking linear 5′-thiol-derivatized PNM to maleimide-derivatized RC or by cross-linking RC and PNM by reaction of RC with 2-iminothiolane followed by addition of the PNM and mild ultraviolet irradiation. The last method is recommended in cases where other methods are inappropriate because it leads to a complex mixture of reaction products of which the optimum average cross-linkage is tedious to establish and can moreover be difficult to standardise between badges.

In a preferred embodiment of this invention, the RC and PNM are coupled by condensing PNM, based on its overall negative charge, onto RC-poly-cation conjugates, that have an overall positive charge due to the poly-cation. The advantage of such electrostatic interaction is that the DNA is not covalently coupled to the RC and, once in the host cell, can dissociate from the RC-poly-cation conjugate. Polycations that can be used, for tight complexing of PNM with the RC-poly-cation conjugate, are polypeptides such as poly-lysine, poly-arginine, poly-ornithine and basic proteins such as histones, avidin and protamines.

Depending on the type and quantity of carbohydrate moieties on the surface of the RC (and thus on the source of RC), different coupling reactions with poly-cations are reconunended. For example, if carbohydrate moieties on the RC carry accessible sialic acid groups (N-acetylneuraminic acid), coupling of the RC complex with a polycation such as poly-lysine is possible in a two-step procedure based on selective oxidative modification and activation of the sialic acids on the carbohydrates, followed by reductive coupling to the poly-lysins amino groups. Within the branched carbohydrate chains, sugars that contain a vicinal diol system can be oxidised by sodium periodate to yield aldehydes, with the concomitant cleavage of the HOC—COH bond. To evaluate whether terminal exocyclic carbon atoms of sialic acids within the carbohydrate chains are effectively removed by periodate oxidation, one can check whether tritium is indeed selectively incorporated into the modified sialic acid residues as described by Kisllimoto T. & Tavassoli M., 1986. The skilled man will know to select and adapt protocols such as to guarantee that upon coupling of the RC to the PNM, the ribosomal subunits of the RC preserve, to a substantial extent, their integrity.

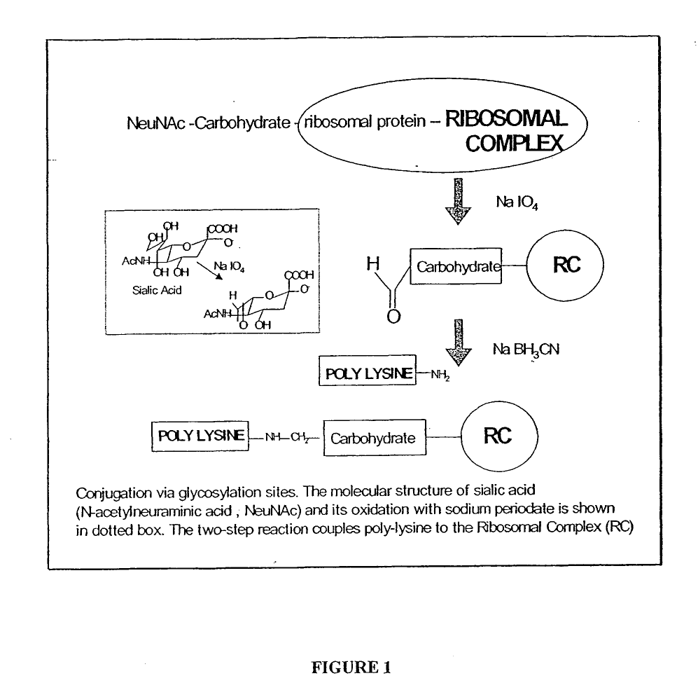

In a specific embodiment, the poly-cation used is poly-lysine and the RC contains carbohydrate moieties with accessible sialic acid groups that upon oxidation with NaIO₄ can be coupled via the freed aldehyde group to the N-terminal amino-group of poly-lysine. The junction that results from aldimine formation is stabilised by reduction to the secondary amine with sodium cyanoborohydride, which is no longer hydrolysable (see FIG. 1). This procedure can be done as a two-step procedure:

1. Conjugation of RC via an oxidised carbohydrate moiety to poly(L-lysine): Ribosomal Complex (RC) from a selected Microbe is prepared as described earlier in this invention disclosure. Poly(L-lysine) can be obtained from SIGMA. A solution of 100 mg of RC in 8 ml of 30-mM sodium acetate buffer (pH 5) complemented with 10 mM MgCl₂, is subjected to gel filtration. The resulting solution (approximately 5.7 ml) is cooled to 0° C. and 120 μl of 30 mM sodium acetate buffer (pH 5), containing 6 mg of sodium periodate and 10 mM MgCl₂ is added. The mixture is kept in an ice bath and in the dark for 90 min. For removal of low molecular weight products an additional gel filtration step can best performed. This operation should yield 70% to 80% of oxidised RC. This can be monitored by evaluating the change in UV absorption values before and after, at 280 nm. In order to detect the oxidised aldehyde-containing form that gives a colour reaction upon staining with anisaldehyde reagent, samples can be spotted on a silica gel thin layer plate, dried, immersed into p-anisaldehyde/sulfuric acid/ethanol (1/1/18), dried, and heated.

The oxidised RC solution is promptly added (within 10-15 min) to a solution containing 0.50 μM of poly (L-lysine) with an preferentially an average chain length of 300 lysine monomers in 4.5 ml of 100 mM sodium acetate (pH 5) and 10 mM MgCl₂, with vigorous mixing at room temperature. Optimal chain length of poly (L-lysine) may vary, but is generally between 200 and 400 lysine units and is to be determined empirically in comparative experiments. For calibration experiments, it is desirable to use fluorescent labelled poly (L-lysine). The labelled poly (L-lysine) can be derived by reacting 34 mg of hydrobromide salt with 130 μg of fluorescein isothiocyanate (FITC) in sodium bicarbonate buffer (pH 9) for 3 h and subsequently gel filtration. Poly-lysine content of fractions can then be estimated spectrophotometrically by absorption at 495 nm (Kontron SFM25 fluorescence detector). The amount of dithiopyridine linkers in the modified RC can be determined after reduction of an aliquot with dithiothreitol followed by absorption measurement of released pyridine-2-thione at 340 nm.

After 20 min, the pH of the mixed solution is brought to 7.5 by addition of 1M sodium bicarbonate, 0.01M magnesium chloride; four portions of 14.2 mg (±150 μm) of sodium cyanoborohydride each are added at 1-h intervals. After 18 h, 2.8 ml of 5-M sodium chloride, 0.01M magnesium chloride are added to bring the solution to an overall salt concentration of about 0.75-M. The reaction mixture is next loaded on a cation-exchange column (Pharmacia Mono S HR 50/50) and is fractionated with a salt gradient from 0.75 to 2.5 M sodium chloride, with constant content of 25 mM 4,(2-hydroxyethyl)-1-piperazineethanesulfonic acid (HEPES) and 10 mM magnesium chloride. The high salt concentration during the loading of the column and at the beginning of the gradient enhances the recovery of the polycation conjugates. Elution can be followed by fluorescence. Fractions containing most poly (L-lysine)-RC conjugate are pooled and dialysed against 2×2 L of 25 mM HEPES (pH 7.3), 10 mM Magnesium chloride. Overall yield of these conjugates, as measured based on either RC or on polylysine coupling (comparison with starting quantities), can be higher than 50% for either. RC conjugates can be stored (after shock-freezing in liquid nitrogen) at −20° C.

2. Polynucleotide Molecule (PNM) complexation with poly(L-lysine)RC conjugate (PL-RC): In this preferred embodiment the PNM consist of plasmid DNA, although PNM of other nature can also be used. For use of Immunogenic Complex in vaccines, the PNM contains a transcription unit encoding the Antigen gene or Antigen polynucleotide sequence of interest. However, for calibration purposes of relative amounts of PNM versus PL-RC, one can insert a Photinus pyralis luciferase gene cassette in the vector. Firefly luciferase is a well-known eukaryotic gene expression marker, useful for evaluation of gene expression in transfected host cells. Expression levels of different preparations of Immunogenic Complex (IC), each with a different ratio of PNM to PL-RC, can be compared in order to select the optimal ratio. A simple protocol for preparation of IC, based on PNM complexed with PL-RC, is as follows: 5 mg of plasmid DNA [containing a DNA transcription unit] in 33 ml of HBS (150 mM NaCl, 10 mM MgCl₂, 20 mM HEPES, pH 7.3) are mixed with 50 mg of PL-RC (prepared as described above) in 17 ml of HBS. Phosphate buffers are not preferred as precipitation may occur. Complexation of PNM with PL-RC of this 50-ml solution is allowed to occur for 30 minutes; subsequently the solution is fractionated in 100 μl aliquots, snap frozen by liquid nitrogen and stored at −20° C. until further use. Good condensation of the DNA on the poly-cation conjugated RC is important for efficient delivery and uptake of the PNM by the target host cells. DNA condensation can be verified using a JEOL 100S electron microscope (EM) operated at 80 kV. Rotary shadowing can be done on IC samples (typically containing 2 μg of DNA complexed with approximately 6 μg PL-RC). These IC samples are suspended in 100 μl of 150 mM ammonium acetate, 10 nM MgCl₂ (instead of HBS), supplemented with 2 μl of 0.3% isoamyl alcohol/0.2% cytochrome c, mixed with equal amounts of glycerol. Samples are sprayed onto freshly cleaved mica plates and shadowed with carbon/platinum as described by Tyler, J. M. & Branton D., 1980. Well-condensed structures show condensed globular or ring-like structures. Less condensed structures show a more “woolly” knot phenotype.

In cases where the RC does not or does not sufficiently carry carbohydrate moieties with accessible sialic acid groups, that are accessible for conjugation in a reproducible fashion, then the selected poly-cation, preferentially poly (L-lysine), can be covalently conjugated with ribosomal proteins of the RC utilising the well-established carbodiimide method according to Williams & Chase, Methods of Immunology and Immunochemistry, 1967,1:155-156.

In another preferred embodiment of this invention, the RC and DNA-based PNM are covalently coupled in a 4-step procedure: (1) the RC is derivatized with maleimide groups, (2) an 5′thiol derivatized oligonucleotide linker is prepared, (3) this linker is ligated via its non-modified end to PNM (4) the 5′thio-derivatized PNM is coupled via the freed thiol-group to maleimide-derivatized RC. This can be done as follows:

1. Synthesis of 5′thiol derivatized linker A short 10 to 20 nucleotide self-complementary linker 5 is made carrying a 5′-5′ linked uridine moiety on one end and containing on the other end, an cleaved enzyme restriction site corresponding to a selected enzyme restriction site on the PNM, that is not located in DNA transcription unit and is preferentially unique or low abundant in the PNM. It can be prepared using a fully automated DNA synthesiser (Pharmacia, Gene Assembler). The standard dimethoxytrityl nucleoside phosphoramidite coupling method can be used on a 10 μmol controlled-pore glass support column (Matteucci M. D. & Caruthers M. H., 1981).

For synthesis of the 5′thiol-derivatized oligonucleotide, introduction of the 5′-5′ linked uridine moiety can be accomplished using 2′3-di-O-acetyluridine 5′-(2-cyanotethyl N,N-diisopropylplhosphoramidite). This reagent is prepared starting from known 2′,3′-di-acetyluridine by phosphitylation with (2-cyanoethyl)-N,N-diisopropylaminochlorophosphine (Slina N. D., et al., 1984). After completion of the solid-phase synthesis, cleavage of the oligonucleotide from the solid support and removal of protecting groups is carried out by incubation in saturated dry ammonia/methanol solution (20 ml) in a sealed flask for 72 h at 50° C. The support is removed by filtration and the filtrate is evaporated under reduced pressure. The crude unprotected DNA fragments are chromatographed on Sephadex S-100 (HiLoad HR) 2 cm²×150 cm) with 0.05 M triethylammonium bicarbonate (TEAB) buffer (pH 7.8). The appropriate fractions (as monitored with HPLC) are pooled, concentrated to a small volume, and lyophilized. The oligonucleotide is then converted into the Na⁺ form by passing through a column of Dowex 50W X4 cation-exchange resin. The resulting UV-positive fractions are pooled and again lyophilized. Purity can be confirmed by HPLC analysis. Analytic HPLC can be conducted on a Waters 600E (system controller) single-pump gradient system, equipped with a Waters Model 484 variable wavelength UV detector and a Waters Model 741 data module under following conditions: Pharmacia FPLC ion-exchange column MonoQ HR 5/5; buffer A, 0.02 M K H₂PO₄ (pH 5.5, 25% acetonitrile); buffer B, 0.02 M KH₂PO₄ and 2.0 M KCL (pH 5.5, 25% acetonitrile); gradient, 0-20 min linear 0→64% B; flow rate 1.0 ml/min; detection at 254 nm.

To thio-protect the 5′-5′ uridine moiety, 0.05M NaIO₄ (sodium iodate) in 125 μl 0.1 M ice-cold sodium acetate buffer (pH 4.75) is added to a solution of this oligonucleotide (0.5 mM corresponding to 2-2.5 mg) in the same acetate buffer. Next, 100 μl methanol is added to clear the solution. The reaction mixture is incubated for 45 min. at 0° C. in the dark, followed by chromatography on a Sephadex G-10 column (1 cm×10 cm) using water as eluent. DNA-containing fractions are pooled and concentrated to a volume of ca. 50 μl. To this solution is added, respectively, 0.1 M phosphate buffer (pH 8.0)/methanol (2:1 v/v) (100 μl), 0.04 M S-pyridylcysteamine hydrochloride in the same phosphate buffer/methanol mixture (30011) and, after 30 min, freshly prepared 0.1 M sodium cyanoborohydride in methanol (25 μl). The clear solution with a final pH of 8.0 is incubated overnight at room temperature. An additional portion of freshly prepared sodium cyanoborohydride (0.1 M in methanol, 50 μl) is added and the reaction mixture is incubated for another 90 min at room temperature. After removal of methanol by evaporation, reagents are separated from derivatized DNA by chromatography on Sephadex G-25 (1 cm×45 cm) using 0.05 M triethylammonium bicarbonate (TEAB) buffer (pH 7.8). The elution profile can be monitored by UV absorbance at 254 nm. The fractions containing DNA, as established by HPLC (MonoQ), are collected and lyophilized twice with water. The oligonucleotide is stored at −20° C. In order to determine the S-Pyridyl content of the thiol-derivatized oligonucleotide, a sample is dissolved in 0.1 M phosphate buffer (pH 8.0)/methanol (2:1 v/v) to a concentration of ca. 20 μM. The oligonucleotide concentration is determined by measuring absorbance at 260 nm (ε₂₆₀=126.000 M⁻¹×cm⁻¹). To this solution is added 50 mM dithioerythritol (DTE) in the above phosphate buffer/methanol mixture to a final concentration of 500 μM. The difference in absorbencies at 343 and 400 nm (reference) is determined before and after DTE addition. The increase of this value is ascribed to release of pyridinethione (ε₃₄₃=8080 M⁻¹×cm⁻¹).

Synthesis of the complementary oligonucleotide strand which is not 5′ thiol-derivatized is conducted in similar fashion (except thiol-protection), but has as first 3′ end deoxyribonucleotide a nucleotide complementary to the first 5′end desoxynucleotide following the 5′-5′linked uridine moiety and has at its 5′end deoxyribonucleotides such that upon hybridization with the 5′thiol-derivatized oligonucleotide, the desired (protruding) cleaved restriction enzyme end is composed.

Duplexes are made by mixing equimolar amounts of the two complementary strands to a concentration of 1×10⁻⁵ M per strand in 0.01 M Tris-HCL (pH 7.0) adjusted to 0.1 M sodium concentration with NaCl.

2. Ligation of the Thiol-derivatized duplex oligonucleotide with Polynucleotide Molecule: The Polynucleotide Molecule (PNM) is suspended in appropriate restriction buffer and cleaved with two selected restriction enzymes which do not cleave the DNA transcription unit and whereby one enzyme produces a restriction enzyme cleavage site corresponding to the cleaved restriction enzyme site at one end of the thiol-derivatized duplex oligonucleotide and a second restriction enzyme which provides different, non-compatible restriction sites, and preferentially chosen such as to provide DNA fragments of sufficient different sizes to easily separate the fragment carrying the DNA transcription unit by preparative scale DNA fragment purification. The DNA fragment harboring the DNA transcription unit now carries two differently restricted ends. It is preferentially purified from the other DNA fragments and carries at one end, a cleaved (protruding) restriction site which can be ligated to the thiol-derivatized duplex oligonucleotide. Once resuspended the DNA fragment containing the DNA transcription unit can be spectrophotometrically quantified by UV absorbance at 260 nm. This DNA fragment is diluted in ligation buffer with the thiol-derivatized duplex oligonucleotide (minimum 100× molar excess) and ligated. The resulting product is a PNM which is thiol-derivatized at one end.

3. Derivation of Ribosomal Complex (RC) with maleimide groups: Ribosomal proteins of the RC are derivatized with maleimide groups by treatment with succinimidyl-4-(N-maleimidomethyl)cyclohexane-1-carboxylate (SMCC) such that the appropriate average number of maleimide groups (0.5-10) are formed per RC.

The preparation of maleimide-derivatized RC can be achieved as follows: To 50 mg of RC in phosphate buffer (0.05M, pH7.5) containing 0.01M MgCl₂ (PB) in 2.5 ml is added 0.01 M SMCC. The reaction mixture is incubated for 90 min. at room temperature in the dark. Excess SMCC is removed by gel filtration on Sephadex G-25 (PD-10 column, Pharmacia) in 50 mM phosphate buffer (pH 6.0) containing 0.01 M MgCl₂, 0.1 M NaCl and 5 mM EDTA. The concentration of RC in the resulting solution can be determined by measuring absorbance at 280 nm using a Waters Model 484 variable wavelength UV detector.

The number of maleimide groups incorporated onto the RC is determined as follows: a small portion (500 μl) of maleimide-derivatized RC is reacted with 50 mM cysteamine (200 μl) in the phosphate buffer (pH 6.0) for 10 min. Unreacted cysteamine is back titrated with 5,5′-dithiobis(2-nitrobenzoic acid) (DNTB) and quantified spectrophotometrically at 412 nm after 15 min. (ε₄₁₂=13.600 M⁻¹×cm⁻¹). As a reference cysteamine oxidation with DTNB in the absence of maleimide is determined. By varying the SMCC concentration, one can establish different average numbers of maleimide groups per RC.

4. Conjugation of Maleimide-derivatized RC with 5′thiol-derivatized PNM: In principle, each maleimide group on the derivatized RC can form a conjugate, via a stable thioester linkage, with the free thiol group on one 5′end of the PNM, producing an IC consisting of a RC onto which linear PNM's are attached by one end.

Conjugation of maleimide-derivatized RC with 5′thiol-carrying PNM can be achieved as follows: The 5′ thiol-protected PNM is dissolved in 0.1 M phosphate buffer (pH 8.0)/methanol (2:1 v/v), which had been thoroughly degassed with nitrogen, to a concentration of 250 μM. Under a nitrogen atmosphere, 5 mM tributylphosphine in 2-propanol is added (0.75 equiv. with respect to the total amount of DNA, i.e. equimolar relative to the pyridyl disulfide present). The reduction is allowed to proceed for 15 min. a room temperature. Meanwhile a solution of maleimide-derivatized RC (25 mg) in buffer containing 50 mM phosphate, 0.1 M NaCl, 0.01 M MgCl₂, and 5 mM EDTA at pH 6.0 is also degassed and the reduced DNA solution is added under nitrogen to the functionalized RC. Conjugation is allowed to proceed overnight at 4° C. Finally, unreacted maleimide groups are blocked by addition of cysteamine. (0.01M) in water. Chromatography is performed at 4° C. and the eluate is monitored at 280 min. The fractions containing RC are collected and can be stored at 4° C.

The conjugation ratio can be determined as follows: The number of PNM conjugated to RC can be quantified spectrophotometrically by determination of the absorbance of conjugate in PBS both at 260 nm and 280 nm, given that the extinction coefficients of RC and PNM are determined. Values for unreacted RC and unreacted PNM can be empirically established and are here called respectively X and Y. The relation can be derived between A₂₆₀/A₂₈₀ (measured) and the conjugation ration (Z) is as follows:

$$Z = \frac{{ϵ}_{RC , 280}}{{ϵ}_{DNA , 260}} \times ( \frac{{A}_{260} / {A}_{280} - X}{1 - ( {A}_{260} / {A}_{280} ) / Y} )$$

Preferentially the resulting IC consist of RC and PNM whereby an average of 0.5 to 10 PNM are linked to the RC.

Yet another primary aspect of the present invention concerns the use of the Immunogenic Complex in pharmaceutically acceptable prophylactic vaccines that may be delivered without injection or ballistic methods to the Mucosal Immune System of oral, nasal, bronchial, esophageal, gastro-intestinal, rectal, vaginal mucosa as well as those of ear and eye.

In one embodiment of the present invention, the Immunogenic Complex is the active principle in prophylactic combination vaccines against microbial species and viruses.

In another embodiment of the present invention, the Immunogenic Complex containing Ribosomal Complex from multiple Microbes and Polynucleotide Molecule from either Microbe or virus, has therapeutic use against microbial infection, in disease management and in other cases where stimulation of the immune system is desirable.

Bacteria, Fungi and Protozoa from which Ribosomal Complexes and/or Polynucleotide Molecules can be prepared, for use in Immunogenic Complex or Heterologous Immunogenic Complex, include, but are not limited to the following under lists 1, 3 and 4 in the table below; Viruses from which Polynucleotide Molecules can be prepared for use in Bacterio-viral Immunogenic Complex include, but are not limited to the following under list 2 of the table below:

List 1: bacteria
Actinobacillus
actinomycetemcomitans
Bacille Calmette-Guérin
Bordetella pertussis
Campylobacter consisus
Campylobacter recta
Capnocytophaga sp.
Chlamydia trachomatis
Eikenella corrodens
Enterococcus sp.
Escherichia coli
Eubacterium sp.
Haemophilus influenzae
Klebsiella pneumoniae
Lactobacillus acidophilus
Listeria monocytogenes
Mycobacterium tuberculosis
Mycobacterium vaccae
Neisseria gonorrhoeae
Neisseria meningitidis
Nocardia sp.
Pasteurella multocida
Porphyromonas gingivalis
Prevotella intermedia
Pseudomonas aeruginosa
Rothia dentocarius
Salmonella typhi
Salmonella typhimurium
Serratia marcescens
Shigella dysenteriae
Streptococcus mutans
Streptococcus pneumoniae
Streptococcus pyogenes
Treponema denticola
Vibrio cholera
Yersinia enterocolitica
List 2: viruses
Coxsackievirus
Cytomegalovirus
Denque virus
Hepatitis A virus
Hepatitis B virus
hepatitis C virus apthovirus
herpes simplex virus
human immunodeficiency virus
infectious bronchitis virus
influenza virus
Japanese encephalitis virus
papillomavirus
porcine transmissible gastro-enteric virus
respiratory syncytial virus
rotavirus
rubella virus
suipoxvirus
vacciniavirus
varicellovirus
variolavirus
virus parainfluenza virus rhinovirus
yellow fever virus
List 3: fungi
Candida albicans
Blastomyces dermatitidis
List 4: protozoa
Plasmodium falciparum
Trypanosoma cruzi
Leishmania sp.
Entamoeba histolitica

Pharmaceutical compositions can be prepared for prevention and treatment of infectious disease caused by Microbes. Such pharmaceutical compositions comprise Immunogenic Complex wherein the Immunogenic Complex is formulated with pharmaceutical carriers in pharmaceutically acceptably delivery forms such as liquids, aerosols, lyophilised powders, pills, creams and suppositories; some of which may contain compounds such as erythrosine, titanium dioxide, Fe₂O₂, D-mannitol, magnesium stearate, gelatine, oils, waxes, antibiotics or antiseptica for administration to animals and/or humans.

The dosage and route of administration depends to a large extend on the condition and weight of the subject being treated, as well as on the frequency of treatment. The response of the initial prime inoculation and clinical judgement of the effect may influence regiments for boost immunisations, including dose. While the above described Immunogenic Complex may be produced and formulated for injection (parenteral or intramuscular), it is particularly suited for delivery to mucosal tissues of nose, mouth and throat by spray of a liquid suspension, delivery to upper respiratory tract by dry or liquefied aerosol spray, delivery to the gastrointestinal tract in protective matrix or microparticle, formulated in a pill, and delivery to rectal and vaginal mucosa incorporated in a gelatinous capsule or suppository.

While the invention has been described and illustrated herein by references to the specific embodiments, various specific material, procedures and examples, it is understood that the invention is not restricted to the particular material combinations of material, and procedures selected for that purpose. Indeed, various modifications are intended to fall within the scope of the appended claims.

It is further to be understood that all base sizes or amino acid sizes, and all molecular weight or molecular mass values, given for nucleic acids or polypeptides are approximate, and are provided for description.

The following examples are offered by way of illustration and are not intended to limit the invention in any matter.

### EXAMPLE 1

### Preparation, Administration and Evaluation of a Vaccine Against Bordetella Pertussis, Causal Agent of Whooping Cough, Based on Immunogenic Complex

Whooping Cough is a highly contagious human respiratory disease, typified by episodes of paroxysmal coughing. It is caused by Bordetella pertussis, which infects ciliated, respiratory epithelia of the nasopharynx, trachea and bronchial tree. Filamentous hemagglutinin (FHA) is an Adhesin involved in mediating the interaction of B. pertussis with human cilia. Existing vaccines are of limited efficiency, probably because they only induce a strong humoral IgG response and do not produce sufficient pathogen-clearing IgA's at the mucosal surfaces. Whooping cough is considered a major health hazard of very young infants, elderly, cystic fibrosis patients and immuno-compromised patients.

1. Culture of Bordetella pertussis: Streptomycin-resistant or gentamycin-resistant strains, such as for example IPbp0405, can be recovered from cough sample of a sick infant diagnosed with pertussis. IPbp0405 is streptomycin resistant and can be grown on Bordet-Gengou agar supplemented with 1% glycerol and 20% defibrillated sheep blood (BG) or in modified Stainer-Scholte medium containing 2,6-O-dimethyl-β-cyclodextrin at 1 g/litre. For selection purposes, streptomycin is added at 100 μg/ml to IPbp0405 cultures.

2. Preparation of Ribosomal Complex (RC) from B. pertussis: RC can be prepared essentially as described earlier in this invention disclosure. RC is resuspended in a phosphate buffer (0.01M, pH 7.0) containing 0.01 M MgCl₂ (PMB), quantified (on basis of ribosomal protein content) and stored frozen at −80° C.

3. Cloning of the fhaB gene of B. pertussis in pBen0161: The 220 kDa mature FHA protein is derived from a 370 kDA precursor protein. The corresponding fhaB gene lies, with the exception of approximately 200 bp at the 3′ end, entirely on a EcoR I-Bcl I restriction fragment (see FIG. 2). The lacking 3′ fragment can be amplified, based on information of GenBank fhaB sequence data (NCBI accession # M60351), using primers which render the 3′ fhaB fragment a 5′ Bcl I and 3′ BamH I site. After reconstruction of the entire gene by ligation (Bcl I site) of the major fhaB containing fragment and the small 3′ fhaB fragment, the resulting EcoR I-BamH I fragment can ligate to corresponding EcoR I & BamH I restriction sites of pUC19 poly-linker (NCBI accession # X02514) forming a plasmid called pUC19-fha. This plasmid carries the fhaB gene in reverse orientation vis-á-vis the LacZ promoter. pUC19-fha is subsequently linearized at the EcoR I site and aliquots of the DNA are subjected to BAL-31 exonuclease activity treatment over different time periods, removing the 5′ untranslated sequence of the fhaB gene fragment. Restriction with BamH I liberates the trimmed fhaB fragments, which are treated with mung bean nuclease to remove protruding ends; after enzyme inactivation, the fragments are subsequently digested with a restriction enzyme which only cleaves within the ampicilin resistance gene (to avoid vector reconstitution). The resulting mixture is cloned into the Sma I site of pBen0161 and ligation products are transformed into E. coli. Ampicilin resistant colonies are isolated and used for DNA preparation, restriction digestion and DNA sequence analysis in order to identify the clones that contain plasmid containing the full length fhaB gene in the proper orientation such that the DNA transcription unit contains the human hemoglobulin control region at the 5′ of the fhaB gene and the rabbit β-globin translation termination sequences at the 3′ end. The appropriate resulting plasmid is called pBen220.

4. Preparation of Polynucleotide Molecules consisting of pBen220: The plasmid pBen220, harbouring the fhaB gene is transferred to E. coli strain DH5a. Preparative scale plasmid DNA can be prepared as described earlier in this disclosure.

5. Covalent linkage of 5′thio-derivatized Polynucleotide Molecule (PNM) via freed thiol-group to maleimide-derivatized Ribosomal Complex (RC) of Bordetella pertussis: As described earlier in this invention disclosure, a short (15 bp in length), nucleotide self-complementary linker is made and carries a 5′-5′linked uridine moiety on one side and a cleaved restriction enzyme site (corresponding to a selected enzyme restriction site outside the fhaB gene on pBen220) on the other side. Here the Bsa I site, which occurs at the 3′ side of the ampicilin resistance gene (Ap) on pBen220, can be used. The PNM is cleaved with Bsa I and with a second enzyme creating non-compatible restriction ends (with Bsa I site), for example the Xmn I site, which cuts at the 5′ side of Ap on pBen220. Ligation of the 5′thiol-derivatized duplex oligonucleotide with the double-digested PNM generates a PNM that is thiol-derivatized at one end.

The ribosomal proteins of RC of B. pertussis are derivatized with maleimide groups by treatment with SMCC, as described earlier in this invention disclosure. SMCC concentration is varied for empirical evaluation and the preparative concentration chosen such that approximately 2 to 4 maleimide groups are made per RC.

Conjugation of maleimide-derivatized RC with 5′-thiol-carrying PNM can be achieved as described earlier in this disclosure and such that each RC carries on average 2 PNM. The collected fractions of Immunogenic Complex (IC) can be brought to 0.1 M phosphate buffer (pH 7.5) supplemented with 5 mM EDTA and 10 mM MgCl₂, and are stored frozen at −80° C.

6. Immunisation and infection of mice: Ninety 5-week old BALB/cAnNcR mice can be used. Animals are housed in pathogen-free isolation cubicles at a constant 24° C. and 12 h day/night cycle. The mice are fed mouse chowder ad libitum. One week after arrival in the laboratory immunisations of each group of 14 mice can be done with respectively following antigens: [1] IC (10 μg), [2] RC of B. pertussis (5 μg), [3] pBen220 DNA (5 μg), [4] Filamentous Hemagglutinin (FHA) (5 μg), [5] BSA (10 μg) (negative control group). FHA is purified from B. pertussis strain IPbp0405 and represents a single band of 220 kDa on sodium dodecyl sulphate (SDS)-polyacrylamide gels and has less than 0.001% pertussis toxin contamination as determined by the Limulus amoebocyte lysate assay. Mice are briefly anaesthetised with Metofane inhalant anaesthesia (Pitman Moore, Chicago, Ill. USA) Immunisation are done by intranasal administration (all quantities in 10 μl sterile physiological saline per nostril, done twice) by means of a pipettor with sterile disposable tips; mice are held upright for a little while to assure that the dose is well inhaled. One week after arrival in the lab, a positive control group [6] consisting of 20 mice, is put into an 0.27 m³, air-tight container with a medical ultrasonic aerosol inhaler apparatus loaded with 50 ml of 10⁹ CFU/ml of B. pertussis in sterile phosphate-buffered saline (PBS). Mice are removed from the aerosol chamber after 1 h. Two mice are immediately sacrificed to determine the number of viable B. pertussis cells in their lungs. For this purpose, lungs and tracheas are aseptically removed and homogenised in sterile PBS, and dilutions are plated on Bordet-Gengou agar to determine the number of recoverable bacterial colonies. Appropriate inoculation has occurred if the infected animals have approximately 10⁵ CFU in their lungs. Seven and fourteen days after the first administration, mice of groups [1] to [5] are given booster immunisations of same quantities in 10 μl physiological saline per nostril, administered twice). At each of these time points, 2 mice of group [6] are sacrificed for evaluation of lungs and recovery of B. pertussis CFU.

Four weeks post the first immunisation, 5 mice from each group [1] to [5] are infected with B. pertussis, whilst 9 mice of group [6] are re-infected according to same method as described above. Five weeks and 6 weeks post first immunisation, 2 mice of each group [1] to [5] and of the 9 re-infected mice of group [6] are sacrificed for evaluation of lungs and recovery of B. pertussis CFU. All experiments are best conducted at least twice and data values added and averaged.

7. Sampling of serum and respiratory Immunoglobulins: On the day prior to challenge of the mice with B. pertussis, 5 mice of each group [1] to [6] are anaesthetised with tribromoethanol and are bled from the brachial artery. The serum is separated by centrifugation and pooled for each group of animals; their tracheas can be cannulated with a piece of PE-50 polyethylene tubing (Clay Adams, Parsippany, N.J., USA) that is held in place with a tied loop of suture. Sterile PBS (0.5 ml) is gently instilled into the lungs and very slowly withdrawn three times. The resulting broncho-alveolar fluids (BAF) of mice of each group are pooled, are centrifuged and the supernatants are removed and frozen at —20° C. until analysis. Four weeks post challenge with B. pertussis, 5 remaining mice of groups [1] to [6] are also bled for serum and have BAF collected.

8. Immunoassays and enzyme-linked immunosorbent assay (ELISA) for respectively serum- and BAF-specific IgG and IgA: All immunoassays are performed with the pooled serum and BAF's of each group. B. pertussis lysate is obtained by suspension of end-log 1 ml culture in an equal volume of Tris-magnesium-0.05 M NH₄ buffer [0.05 M NH₄Cl, 0.01 M magnesium acetate, 0.01 Tris-HCL (pH 7.4)] and passage through a French press twice at 16.000 lb/inch². The lysate is diluted 10×m in coating buffer (carbonate-bicarbonate buffer, 50 mM, pH 9.4) and used to coat the appropriate 96 well micro-titer plates (NUNC, Polysorb immuno Plates). After 2 hours of incubation at 37° C., the remaining binding sites on all plates are blocked for 30 min. with phosphate-buffered saline (PBS) supplemented with 0.2% (vol./vol.) Tween 20 at room temperature. Subsequently 100 μl of serum or 50 μl of BAP is added per well in two-fold dilutions in ELISA dilution buffer and is incubated for 1 hour at 37° C. Thereafter, plates are treated for 1 hour at 37° C. with optimal dilutions of respectively horseradish peroxidase-conjugated goat anti-mouse IgG- or IgA (Boehringer-Mannheim, Del.). The binding can be measured in an ELISA reader (Bio-Rad Labs, Ca, USA). For colour development the substrate, 2.2′-azinobis(3-ethylbenzthiazoline-6-sulfonic acid) (ABTS), is added and the optical density is spectrophotometrically measured at 405 nm (OD405) after 15, 30 and 60 minutes incubation at 37° C. Two other sets of microtiter plates are coated respectively coated with 5 μg purified FHA per ml ELISA coating buffer and 5 μg purified B. pertussis fimbriae type 2, for 3 hours and further treated as above.

9. Rcsults:

a) Immune response to the vaccine preparations against Bordetella pertussis, FHA and Fimbriae type 2:Strong response (serum IgG and BAF IgA) to B. pertussis can be observed in mice immunised with Immunogenic Complex of Bordetella pertussis (IC-Bp) (group [1]) and in mice immunised with B. pertussis directly (group [6]), four weeks post immunisation, with inductions 200 to 400 fold higher than in groups inoculated with BSA [5]. Mice inoculated with RC of B. pertussis (RC-Bp) (group [2]) and with Filamentous Hemagglutinin (FHA) (group [4]) also show a good immune induction, whilst pBen220 plasmid DNA provides a weak reaction: approximately 1 log above the negative control group [5], inoculated with BSA. In other words, IC-Bp is effective in induction of both humoral (IgG) and secreted (IgA) antibodies targeted at B. pertussis and is substantially better than RC-Bp alone, while plasmid DNA pBen220 (carrying the DNA transcription unit for FHA) on its own, is weakly immunogenic for vaccination by our liquid nasal delivery protocol. The immune response, measured by induction of specific serum IgG's and BAF IgA's against purified FHA, is also very pronounced in groups [1], [4] and [6] with titers 2 to 3 log's above BSA inoculated control group [5]). Relative to the values of groups [1], [4] and [6], immune induction by RC-Bp (group [2]) against FHA is lower than against total B. pertussis lysate, suggesting a more complex antigen-recognition spectrum of antibodies induced by RC-Bp versus those induced by the antigen FHA. Similar to the ELISA results, using B. pertussis coated plates, pBen220 DNA without carrier/adjuvant is a relatively poor immune inducer against FHA antigen when using this protocol. Finally, when fimbriae type 2 (fim 2) are used to coat ELISA plates, group [6] shows the strongest immune response, followed by JC-Bp and RC-Bp; groups [3], [4] and [5] give no specific immune induction. The above indicates that IC-Bp is a very strong active ingredient for a vaccine, giving the broadest immunogenic spectrum against B. pertussis, only surpassed by inoculation with the virulent pathogen itself.

b) Course of bronchial infection upon inoculation with B. pertussis.

The 2×2 mice of group [6], sacrificed for evaluation of progression of infection and symptoms after respectively I and 2 weeks post inoculation with B. pertussis, show at both timepoints inflammation of the lungs and trachea. Weight gain at the second timepoint is also halted when compared to non-inoculated controls. B. pertussis CFU increase approximately 2 orders of magnitude after I week and show a drop by the second week post inoculation indicating the recovery process of the mice. Of the remaining mice of group [6], reinoculated four weeks post first inoculation and evaluated for symptoms and B. pertussis CFU, it appears that immunity has been achieved as none of the mice sacrificed 1 or 2 weeks post this challenge had substantial increases of CFU. Lung damage is not evaluated as one cannot determine easily whether damage is due to first or second inoculation. Mice of groups [1] to [5] which are challenged with B. pertussis 4 weeks post their first immunisation, are evaluated for symptoms and B. pertussis titers, 1 and 2 weeks later. Mice of groups [1], [2] and [4] show no substantial increase of B. pertussis CFU and show no obvious damage of to lung or trachea epithelia. Mice of groups [3] and [5] however, show disease symptoms similar to those observed with non-immunised mice of group [6], 1 and 2 weeks post their first inoculation. Such results indicate that IC-Bp, IR-Bp and FHA antigens can induce a protective immune response in BALB/cAnNcR mice against B. pertussis infection.

### EXAMPLE 2

### Preparation, Administration and Evaluation of a Vaccine Against Respiratory Diseases Caused by Bordetella pertussis and Respiratory Syncytial Virus, Based on Bacterio-viral Immunogenic Complex

Respiratory syncytial virus (RSV) is the single most important cause of severe lower respiratory tract infection in babies & young children in the US and Europe, and is a serious problem with elderly and HIV patients. Formalin-inactivated RSV has been shown to enhance disease symptoms in seronegative children when subsequently infected by RSV. Simple subunit vaccines focusing on immature envelope proteins have been disappointing, probably because the virus has highly variable surface factors and in addition uses glycosylation to render epitope-binding sites inaccessible to potentially clearing antibodies.

The development of improved vaccines which induce protective immunity against both the bacterial respiratory pathogen, Bordetella pertussis as well as the virus RSV, is highly desirable, in particular as infection by viral respiratory viruses may facilitate subsequent bacterial infection.

1. Preparation of Immunogenic Complex of Bordetella pertussis: This can be performed as described in Example 1.

2. Cloning of the gene encoding the fusion (F) glycoprotein (Fgp)of respiratory syncytial virus (RSN) in pBen0161: The Fusion (F) glycoprotein (Mᵣ=68,000) is located in the viral envelope and mediates fusion of the virion with the target cell. The DNA sequence corresponding to a fusion (F) protein mRNA can be obtained by those skilled in the art, using classical PCR nucleic acid amplification and cloning technology on RNA preparations from RSV-infected Hep-2 cells (ECACC 86030501; European Collection of Animal Cell Cultures, Proton Down, Salisbury, UK). As RSV source, the RSV-A long strain ATCC VR-26 (American Type Culture Collection, Rockville, Md., USA) can be used. Primers to 5′and 3′end can be synthesized based on sequence information of the fusion (F) protein mRNA in genbank-(NCBI accession D00334). Primers are designed such that restriction sites are included at 5′and

3′ of the Fgp gene, allowing directional cloning in pBen061 such that the gene is inserted in proper orientation between the promoter of Human Immunoglobulin Heavy chain and the transcriptional termination sequence of the rabbit β-globin gene. The resulting DNA is transformed into E. coli. Ampicilin resistant colonies are isolated and used for DNA preparation, restriction digestion and DNA sequence analysis in order to identify the clones that contain plasmid containing the full length Fgp gene. The appropriate plasmid is called pBen068.

3. Preparation of Polynucleotide Molecule (PNM): The plasmid pBen068, harboring the Fgp gene is transferred to E. coli strain DH5α. Preparative scale plasmid DNA can be done as described earlier in this disclosure.

4. Preparation of Ribosomal Complex (RC) of B. pertussis: This can be performed as described in Example 1.

5. Covalent linkage of 5′thio-derivatized Polynucleotide Molecule (PNM) via freed thiol-group to maleimide-derivatized Ribosomal Complex (RC) of Bordetella pertussis: Similarly to Example 1, a short nucleotide self-complementary linker is made and carries a 5′-5′linked urindine moiety on one side and a cleaved restriction enzyme site (corresponding to a selected enzyme restriction site outside the Fgp gene on pBen068) on the other side; here the Bsa I site, which occurs at the 3′ side of the ampicilin resistance gene (Ap) on pBen068, can be used. The PNM is cleaved with Bsa I and with a second enzyme (also outside the Fgp gene) creating non-compatible restriction ends (with Bsa I site), for example the Xnm I site, which cuts at the 5′ side of Ap on pBen068. Ligation of the 5′thiol-derivatized duplex oligonucleotide with the double-digested PNM generates a PNM which is thiol-derivatized at one end. The ribosomal proteins of RC of B. pertussis are derivatized with maleimide groups by treatment with SMCC, as described earlier in this invention disclosure.

6. Conjugation of maleimide-derivatized RC with 5′-thiol-carrying PNM (derivatized pBen068): This is achieved as described earlier in this disclosure and such that each RC carries on average 2 or 3 linearized PNM (derived from pBen068). The collected fractions of Bacterio-viral Immunogenic Complex can be brought to 0.1 M phosphate buffer (pH 7.5) supplemented with 5 nM EDTA and 10 mM MgCl₂, and are stored frozen at −80° C.

7. Preparation of Bacterio-viral Immunogenic Complex (BV-IC): In order to prepare the BV-IC, comprizing RC of B. pertussis separately coupled to two different PNM (based on pBen220 and pBen068) and harboring respectively the fhaB gene of B. pertussis and the Fgp gene of RSV, the IC of Bordetella pertussis and the BV-IC comprizing B. pertussis RC and PNM of RSV are quantified as described earlier and mixed in 1/1[w/w] ratio at a concentration of 1 μg/ml in 0.1 M phosphate buffer (pH 7.5) supplemented with 5 mM EDTA and 10 nM MgC)₂.

8. Immunization of mice: One hundred and eight 5-week old BALB/cAnNcR mice can be used. Animals are handled as in Example 1, except where specified differently. Immunizations of groups of 14 mice are done with respectively following vaccine preparations: [1] BV-IC consisting of 50% IC of B. pertussis and 50% of BV-IC comprizing RC of B. pertussis coupled to PNM harboring the Fpg gene of RSV {BV-IC(Bp+RSV)}, (10 μg); [2] IC of B. pertussis {IC-Bp}, (10 μg); [3] BV-IC comprizing RC of B. pertussis coupled to PNM harboring the Fpg gene of RSV {BV-IC(RSV)}, (10 μg); 4] pBen068 DNA (5 μg); [5] BSA (10 μg) (negative control group). Mice are briefly anesthetized with Metofane inhalant anesthesia (Pitman Moore, Chigago, Ill. USA) Immunization are done by intranasal administration (all quantities in 10 μl sterile physiological saline per nostril, done twice) by means of a pipettor with sterile disposable tips; mice are held upright for a little while to assure that the dose is well inhaled.

A positive control group [6] for B. pertussis infection can consist of 20 mice, which are infected one week after arrival in the lab with B. pertussis as described in Experiment I; two mice are immediately sacrificed to determine the number of viable B. pertussis cells in their lungs. A second positive control group [7] for RSV infection can consist of 18 mice, which are infected with RSV, one week and two weeks after arrival in the lab, by administering, upon anesthetization with 2.5 ml of a 4/1 mix of ketamin (Imalgéne 500; Rhone Mérieux, France) and Xylazine (Rompun at 2%; Bayer, France) per kg of body weight, 2 times intranasally (in 50 μl) with 10⁵ 50%-tissue-culture-infectious-doses (TCID₅₀) of RSV.

Seven and fourteen days after the first administration, mice of groups [1] to [5] are given booster immunizations of same quantities in 10 μl physiological saline per nostril, administered twice). At each of these timepoints, 2 mice of group [6] are sacrificed for evaluation of lungs and recovery of B. pertussis CFU.

Four weeks post the first immunization, 5 mice from group [2] are infected with B. pertussis; 5 mice from groups [1], [3] to [5] whilst [5] mice of group [6] are reinfected as in Experiment I. Nine mice from group [7] are reinfected as described above.

Five weeks and 6 weeks post first immunization, 2 mice of each group [1] to [5] and of the 9 reinfected mice of groups [6] and [7] are sacrificed for evaluation of lungs and recovery of respectively B. pertussis CFU or RSV titers. As detection limit quantities, 1.45 log₁₀ TCID₅₀/g lung tissue can be taken as cut-off point. Animal organs can be considered protected when virus titers are reduced by at least 2 log₁₀ relative to BSA-immunized control mouse levels. All experiments are best conducted at least twice and data values added and averaged.

9. Sampling of serum and respiratory immunoglobulins: Sampling of 5 mice of groups [1] to [7] can be done as in Example 1.

10. Immunoassays and ELISA's for respectively serum- and bronchoalveolar fluid (BAF)-specific IgG and IgA: (Extracted largely from Tebbey P. W. et al., 2000, Vaccine 18: 2723-2734).

All immunoassays are performed with pooled serum and BAF's of each group. B. pertussis whole lysate preparations can be done as described in Example I. Fgp protein for coating of 96-well microtiter plates can be prepared as follows: monolayers of Hep-2 cells infected with RSV are scraped off culture plates at 36-48 hr after infection. Cells are sedimented by low speed centrifugation. After washing with phospate-buffered saline (PBS), cell pellets are resuspended in lysis buffer (10 mmol/L Tris HCL, pH 7.6, 140 mmol/L NaCl, 5 mmol/L EDTA, 1% octylglucoside), and the extracts are clarified by centrifugation in a microcentrifuge. Fgp in the clarified supernatants is precipitated with 65% (NH₄)SO₄. The protein pellet, resuspended in 20 mmol/L Tris HCL, pH7.5, 500 mmol/L NaCl, 0.2% octylglucoside can be dialized against the same buffer and immuno-affinity purified using Fgp-specific monoclonal antibodies, prepared as well-know in the art. Anti-Fgp antibody titers were determined as follows: 96-well plates are coated with 20 ng of purified Fgp per well. Serum IgG and mucosal BAF IgA, of pooled samples per group, can be detected by serial titration of samples, three-fold from starting dilution of 1:25 in PBS −0.3% Tween-20, 0.01 M EDTA buffer, pH 7.0. Samples are added to wells for 1 h. After 5 washes in PBS −0.1% Tween-20, plates are treated for 1 hour at 37° C. with optimal dilutions of respectively horseradish peroxidase-conjugated goat anti-mouse IgG- or IgA (Boehringer-Mannheim, Del.). The binding can be measured in an ELISA reader (Bio-Rad Labs, Ca, USA). For color development the substrate, 2.2′-azinobis(3-ethylbenzthiazoline-6-sulfonic acid) (ABTS), is added and the optical density is spectrophotometrically measured at 405 nm (OD405) after 15, 30 and 60 minutes incubation at 37° C.

11. Plaque reduction- and RSV infectivity assays: Antibody neutralising activity can be measured by a plaque reduction assay, using RSV-A long strain and performed on Hep-2 cell monolayers in 96-well microplates. Geometric mean neutralising titers to RSV-A can be obtained for each group ([1], [3]-[5] and [7]) in presence of 5% (vol/vol) guinea pig serum as source of complement (Gen-Trak Inc., Plymouth Meeting, Pa.). Neutralising titers can be expressed as the reciprocal of the dilution which shows a 60% reduction in plaque forming units (pfu) per well compared to virus control wells. To quantitate virus replication, broncho-alveolar fluids (BAF) of mice of each group are prepared as in Example I and are analysed by plaque titration. Data can be presented as the log₁₀ pfu of virus per g tissue.

12. Results:

a) Immune response to the vaccine preparations against B. pertussis, and RSV: Measurements can be made 4 and 8 weeks after initiation of the experiment. Strong response (serum IgG and BAF IgA) to B. pertussis can be observed in mice immunised with Immunogenic Complex of Bordetella pertussis (IC-Bp) (group [2]), and in mice immunised with B. pertussis directly (group [6]), similarly as in Example I. In addition, high titers can also be observed for both specific IgG's as well as IgA's, in mice immunised with BV-IC consisting of 50% IC of B. pertussis and 50% of BV-IC comprizing RC of B. pertussis coupled to PNM harbouring the Fpg gene of RSV {BV-IC(Bp+RSV)} (group [1]). Mice inoculated with BV-IC comprising RC of B. pertussis coupled to PNM harbouring the Fpg gene of RSV {BV-IC(RSV)} (group [3]) also show good IgG and IgA induction, but distinctly lower than in groups [1], [2] and [6]. Mice immunised with BSA (group [5]), pBen068 (group [4]), or RSV (group [7]) showed no significant specific induction of Ig's reactive with B. pertussis.

Results of ELISA reading on Fgp-coated plates: High readings (with means as high as 3 logs above those of the BSA-immunised control group [5]) can be seen, for both serum IgG's and BAF IgA's, with samples of groups [1] and [3]. Highest values are however obtained with the RSV infected mice (group [7]; values of groups [2] and [6] show no Fgp-specific Ig production, whilst group [4] shows low values for IgA's and IgG's, however clearly much lower that the groups [1], [3] and [7].

In other words, BV-IC (Bp+RSV) is effective in induction of both humoral (IgG) and secreted (IgA) antibodies targeted at both B. pertussis as well as RSV. Non of the other vaccine preparations achieve this dual objective. Course of bronchial infection upon inoculation with B. pertussis: Mice immunised with IC-Bp or infected with B. pertussis (groups [2] and [6]) show results similar as those obtained for correspondingly treated mice in Example 1, indicating that IC-Bp antigens can induce a protective immune response in BALB/cAnNcR mice against B. pertussis infection. Course of bronchial infection upon inoculation with RSV: The functional capacity of the immune responses induced by BV-IC(Bp+RSV), BV-IC(RSV) and pBen068 BSA, 4 weeks after infection of mice with RSV and 8 weeks after initiation of the immunisation process, are determined in the plaque reduction assay for neutralising antibody titers. Positive control contains BAF of group [7], which had recovered from RSV inoculation 4 weeks after first infection and was then re-inoculated: 4 weeks later, this group shows strong immune response as no significant development of virus (cut-off point: titers <1.45 log₁₀ TCID₅₀/g lung tissue) can be detected. The negative controls: BAF of mice group [5] (immunised with BSA) and the control sample only receiving the guinea pig complement-containing serum, show 3 to 5 log₁₀ proliferation of virus. Two to three log₁₀ reduction of viral titers versus above negative controls can be observed with BAF of mice groups [1] and [3]. This shows that antibodies secreted in the lungs of mice immunised with BV-IC(Bp+RSV) or with BV-IC(RSV) can control RSV proliferation. In contrast, only a single log reduction is commonly observed with BAF of group [4] (immunised with pBen068 plasmid alone), illustrating the limited immunising capacity of this PNM in our protocol, if not coupled to RC.

The above shows that a Bacterio-viral Immunogenic Complex (BV-IC) consisting of 50% IC of B. pertussis, as described in Example I, and 50% of BV-IC comprising RC of B. pertussis coupled to PNM harbouring the Fpg gene of RSV {BV-IC(Bp+RSV) can induce specific humoral and mucosal responses that are protective against both targeted bacterial and viral pathogens and can constitute the active ingredients of a vaccine that can be delivered nasally.

### EXAMPLE 3

### Preparation, Administration and Evaluation of a Vaccine Against Genital Calidida albicans and Chlamydia trachomatis Infections, Based on Heterologous Immunogenic Complex

In this example, a Heterologous Immunogenic Complex (IC) is prepared that consists of 1/1 mixture of (a) Ribosomal Complex of Candida albicans covalently coupled to Polynucleotide Molecule consisting of plasmid DNA carrying a DNA transcription unit for the C. albicans hwp 1 Adhesin gene and (b) C. albicans Ribosomal Complex coupled to Polynucleotide Molecule consisting of plasmid DNA carrying a DNA transcription unit for the Chlamydia trachomatis SK 59 Antigen gene.

1. Culture of Candida albicans IPca2005: A virulent Candida albicans strain, preferentially isolated from an infected patient is best used as source of components of the Immunogenic Complex. Here, strain IPca2005 (originating from an female individual with chronic vaginal candidiasis) is used. It can be maintained on yeast extract agar. For preparation of Ribosomal Complex, the strain is transferred to liquid culture medium in 6 liter badges, consisting of 1.17% [wt/vol] yeast carbon base, 1% bovine serum albumin (YCB-BSA medium) and cultured in a gyratory shaker (New Brunswick Scientific Co.) at 150 rpm at 27° C. until mid-log phase.

2. Culture of Chlamydia trachomatis: The Chlamydia trachomatis mouse pneumonitis (MoPn) biovar, from the American Type Culture Collection (Rockville, Md. USA) can be used and grown in HeLa-229 cells and prepared as described by Sayada et al., 1991. Stock of the organism are best frozen at −80° C. in aliquots of a solution containing 0.2 M sucrose, 20 mM sodium phosphate (pH 7.2), and 5 mM glutamic acid (SPG).

3. Preparation of Ribosomal Complex: C. albicans cells (in 20 mg wet weight aliquots) are suspended in an equal weight of Tris-magnesium-0.05 M NH₄ buffer [0.05 M NH₄Cl, 0.01 M magnesium acetate, 0.01 Tris-HCL (pH 7.4)] and passed through a French press twice at 16.000 lb/inch². Bentonite is added to a final concentration of 2 mg/ml, and the suspension is centrifuged at 18.000 rpm (30.000 g) for 30 min. at 2° C. in a Sorvall SS34 rotor. The ribosomes are sedimented from the obtained supernatant by a second centrifugation at 40.000 rpm (105.000 g) for 90 min. at 4° C. in a Beckman 50 Ti rotor. The pellet is resuspended in Tris-magnesium-0.05 M NH₄ buffer and the ribosomes are sedimented as before. The pellet is resuspended in Tris-magnesium-0.25 M NH₄ buffer [0.25 M NH₄Cl, 0.01 M magnesium acetate, 0.01 Tris-HCL (pH 7.4)] to give a concentration of 80 OD₂₆₀ units/ml. The ribosomal suspension is clarified by centrifugation at 5000 rpm (2000 g) for 10 min. in a Sorvall SS34 rotor. The clarified ribosomal suspension (approximately 25 ml) is absorbed at 4° C. to a 2.5×50 cm column of DE23 (Whatman) which has been equilibrated with Tris-magnesium-0.25 M NH₄ buffer. The column is washed with 1 liter of Tris-magnesium-0.25 M NH₄ buffer at a flow rate of 300 ml per hour. The ribosomes are then eluted with Tris-magnesium-0.60 M NH₄ buffer [0.60 M NH₄Cl, 0.01 M magnesium acetate, 0.01 Tris-HCL (pH 7.4)] at a flow rate of 200 ml/h. The portion of the column elute containing ribosomes is recognised by its bluish opalescence. More than 80% of the OD₂₆₀ units can be recovered in less than 50 ml of elute. Ribosomal Complex, containing ribosomes dissociated into their 18S and 28S subunits, can be obtained in suspension by dialysis in 0.0 mM MgCl₂, 0.1 M NaCl, and 0.01 Tris-HCl (pH 7.4).

4. Preparation of Polynucleotide Molecule Consisting of Plasmid DNA Carrying DNA Transcription Unit for the Hyphal Wall Protein (HWP1) of C. albicans:

a) Strains and transformation: A virulent C. albicans strain strain such as IPca2005 can be used. The strain is maintained on Sabouraud (SAB) dextrose agar plates (Oxoid Ltd., Basingstoke, UK). For DNA isolation, C. albicans cultures are grown in 100 ml SAB broth for 2 days at 27° C., washed in sterile water, and resuspended in 4 ml of lysis buffer (0.2 M NaCl, 0.4% sodium dodecyl sulphate, 0.1 M Tris-Cl [pH 7.5], 5 mM EDTA [pH 8]). Equal volumes of phenol (pH8) and glass beads are added, and the mixture is vortexed for 10 min. DNA is extracted twice with phenolchloroform-isoamyl alcohol (25:24:1, vol./vol./vol.) at pH 8 and twice with chloroform-isoamyl alcohol (24:1, vol./vol.) and precipitated with 2.5 volumes of ethanol. For extraction of DNA from C. trachomatis, 100 μl of unpurified chlamydial preparation is added to 2.5 ml of a buffer containing 50 mM KCL, 10 mM Tris-HCl (pH 8.3), 2.5 mM MgCl₂, Nonidet P-40 0.45%, Tween 20 0.45% and proteinase K 60 μg/ml and incubated at 56° C. for one hour. Subsequently DNA is extracted, starting with phenol extraction as for C. albicans. DNA concentrations can be determined on a GeneQuant II spectrophotometer. For long-term storage, all stocks can be maintained frozen in 20% glycerol. Competent E. coli cells (XL10-Gold) can be purchased from Stratagene (La Jolla, Calif.) and transformed with a plasmid according to the vendor's instructions. E. coli is grown at 37° C. in Luria-Bertani broth (1% NaCl, 1% tryptone, 0.5% yeast extract).

b) Cloning of the coding sequence of the Hwp1 gene of C. albicans into pBen0161:

This can be done by PCR amplification from total DNA extracted from C. albicans virulent strain IPca2005, using appropriate primer sets based on Hwp I DNA coding sequences (NCBI database, accession number U64206). Standard PCR procedures are well established and fully accessible to the man skilled in the art. The obtained blunt-end fragment is ligated to the linearised and dephosporylated pBen0161 vector, which is described earlier in this disclosure. The plasmid that contains the Hwp1 gene in the right orientation versus the human immunoglobulin gene control region is called pBen016113 (determined after transformation into E. coli). Both DNA strands can be sequenced using well-know DNA sequencing methods such as the dideoxy chain-termination method.

c) Cloning of the coding sequence of the gene for the 59 kDa immunogenic protein SK59 of C. trachomatis into pBen161: This can also be done by PCR amplification from total DNA extracted from a virulent C. trachomatis strain such as EPct1308, using appropriate primer sets based on SK59 encoding gene. sequences (NCBI database, accession number M31119). The blunt-end fragment is ligated to the linearised and dephosporylated pBen0161 vector. pBen016115 is called the plasmid that contains the SK59 encoding gene in the right orientation versus the human immunoglobulin gene control region, as can be determined after transformation into E. coli. Both DNA strands of the SK59 encoding gene can be sequenced in similar fashion as the Hwp1 gene.

d) Preparation of Polynucleotide Molecules containing respectively DNA transcription units encoding HWP1 and SK59 proteins: The plasmids pBen016113 and pBen016115, containing respectively the Hwp1 gene and the gene encoding SK59 immunogenic protein are each separately transferred to E. coli strain DH5α. Preparative scale plasmid DNA can be prepared as described earlier in this disclosure.

5. Conjugation of Ribosomal Complex (RC) of C. albicans via an oxidized carbohydrate moiety to poly (L-lysine): A solution of 100 mg RC is used to conjugate poly(L-lysine) with RC via N-acetyl neuraminic aldehyde-derivatized carbohydrates on the RC. This method is described earlier in this disclosure and the sodium periodate catalysed reaction is illustrated in FIG. 1.

6. Polynucleotide Molecule (PNM) complexation with poly(L-lysine)-RC conjugate (PL-RC): The PL-RC solution prepared above is divided in two equal fractions. To the two fractions are added equimolar quantities of respectively pBen016113 and pBen016115 plasmid DNA, according to the method described earlier in this disclosure. The cationic DNA is allowed to condense onto the anionic PL-RC and the two resulting fractions are quantified and mixed in 1/1 proportion (w/w). The resulting product is Heterologous Immunogenic Complex (HIC) consisting of C. albicans RC onto which is non-covalently coupled PNM carrying respectively the Hwp1 and SK59 encoding genes.

7. Immunisation and infection of mice: Eighty five eight-week old female BALB/c (H-2^{d}) mice can be used. Animals are housed in isolation cubicles at a constant 24° C. and 12 h day/night cycle. The mice are fed mouse chowder ad libitum. One week after arrival in the laboratory immunisations following groups of mice can be done with respectively following antigens: [1] HIC-Ca/Ct (10 μg) {15 mice}, [2] RC of C. albicans (5 μg) {15 mice}, [3] pBen016113 (5 μg) {10 mice}, [4] pBen016115 (5 μg) {10 mice}, [5] BSA (10 μg) {15 mice}, by intranasal administration (all quantities in 10 μl sterile physiological saline per nostril, done twice) by means of a pipettor with sterile disposable tips. Group Seven and fourteen days after the first administration, the mice are given booster immunisations of same quantities in 10 μl physiological saline per nostril, administered twice). Two positive control groups can be included: 10 mice are given 10⁸ C. albicans cells in 20 μl PBS vaginally [6], while another set of 10 mice is given intranasally 10⁴ C. albicans inclusion-forming units (IFU) [7].

5 mice of each group are sacrificed 3 weeks after the first immunisation for recovery of serum. 5 mice of groups [1], [2],[3], [5] and [6] are challenged in the 4^{th} week following first immunisation with vaginal inoculations of 10⁸ C. albicans cells, whilst groups [1, [2], [4], [5] and [7] are challenged with intrabursal inoculation with C trachomatis. Experiments are best done in triplicate; data are pooled and averages calculated. Intrabursal inoculation can be done as follows: animals are anesthesized with methoxyflurane and a lateral abdominal incision is made. The latter set of experimental groups receive 10⁵ C. trachomatis IFU's in 20 μl SPG in the left ovarian bursa and mock-infected HeLa-229 cell extracts, processed by the same protocol used to purify the EB, in the right ovarian bursa according Pal S., et al.,1994.

8. Vaginal cultures: For the isolation of C. albicans and C. trachomatis, vaginal swabs can be collected at regular intervals following the pathogen challenge. For isolation of C. albicans, vaginal fluid is taken from each animal every 2 days for 10 days with a calibrated (1 μl) plastic loop (Dispoinoic, PBI, France), by insertion and removal from the vagina. Fluids can be used to measure vaginal colonisation. To this purpose the content of each loop is vigorously suspended in 0.1 ml of PBS and aliquots are streaked in triplicate on Sabouraud-dextrose agar with chloramphenicol (20 μl ml⁻¹) to calculate the colony-forming units (CFU ml⁻¹) after incubation of the plates at 30° C. for 48 h.

For C. trachomatis, specimens are cultured in McCoy cells, grown in 24-well plates that are centrifuged at 1,000×g for 1 h at room temperature. At 48 h post inoculation, the monolayers are washed with PBS and fixed with methanol. After treatment with a rabbit polyclonal anti-C. trachomatis serum and staining (Pal S., et al.,1994), the chlamyial inclusions are quantified.

9. Immunoassays and enzynme-linked immunosorbent assay (ELISA) for respectively HWP1 and SK59- specific IgG and IgA: As said above, 3 weeks post first immunisations/infections, 5 mice are sacrificed for serum preparation: blood is collected by heart puncture and the serum is separated by centrifugation and pooled for each group of animals. Vaginal washes (collected on regular intervals up to the day of said pathogen challenge) are collected by irrigation of the vagina twice with 20 μl of PBS and are pooled for each group. Similarly, serum and vaginal washes can be collected from the remaining (re)infected mice, in the sixth week post first immunisations/infections.

All immnunoassays are performed with the pooled serum and vaginal washes of each group. C. albicans hyphal lysate is obtained by suspension of end-log 1 ml culture in an equal volume of Tris-magnesium-0.05 M NH₄ buffer [0.05 M NH₄Cl, 0.01 M magnesium acetate, 0.01 Tris-ICL (pH 7.4)] and passed through a French press twice at 16.000 lb/inch². The lysate is diluted 10× in coating buffer (carbonate-bicarbonate buffer, 50 mM, pH 9.4). and used to coat the appropriate 96 well micro-titer plates (NUNC, Polysorb Immuno Plates). EB of C. trachomatis in coating buffer at a concentration of 20 μg/ml is used for coating of another series of plates. After 2 hours of incubation at 37° C., the remaining binding sites on all plates are blocked for 30 min. with phosphate-buffered saline (PBS) supplemented with 0.2% (vol./vol.) Tween 20 at room temperature. Subsequently 100 μl of serum or 50 μl of vaginal wash is added per well in two-fold dilutions in ELISA dilution buffer and is incubated for 1 hour at 37° C. Thereafter, plates are treated for 1 hour at 37° C. with optimal dilutions of respectively horseradish peroxidase-conjugated goat anti-mouse IgG- or IgA (Boehringer-Mannheim, De). The binding can be measured in an ELISA reader (Bio-Rad Labs, Ca, USA). For colour development the substrate, 2.2′-azinobis(3-ethylbenzthiazoline-6-sulfonic acid) (ABTS), is added and the optical density is spectrophotometrically measured at 405 nm (OD405) after 15, 30 and 60 minutes incubation at 37° C.

10. Results:

c) Immune response to the vaccine preparations against Candida albicans HWP 1 Strong response (serum IgG and vaginal IgA) can be observed in groups [1] and [6] with inductions two magnitude orders above titers observed with groups [3] and [5]. Induction by vaccination with RC of C. albicans (group [2]) is lower, but clearly distinct from background (e.g.[5]). In other words, HIC-Ca/Ct is effective in specific induction of both humoral (IgG) and secreted (IgA) antibodies targeted at HWP1, a key adhesin of C. albicans. HIC-Ca/Ct is substantially better than Ribosomal Complex of C. albicans alone, while plasmid DNA pBen016113 (carrying the DNA transcription unit for HWP1) on its own, appears to be insufficiently immunogenic for vaccination by liquid nasal delivery.

d) Immune response to the vaccine preparations against Chlamydia trachomatis SK59 Strong response (serum IgG and vaginal IgA) can be observed in groups [1] and [7] with inductions that are in the range of two magnitude orders higher than titers observed with groups [2], [4] and [5]. In other terms, HIC-Ca/Ct is also effective in specific induction of both humoral (IgG) and secreted (IgA) immunoglobulins targeted to SK59 whilst neither RC of C. albicans nor pBen016115 (carrying the DNA transcription unit for SK59), nor the BSA negative control, induces significant immune response upon nasal inoculation.

e) Course of infection of vagina upon inoculation with C. albicans. The results of vaginal cultures obtained at weekly intervals following vaginal challenge, show that mice inoculated with C. albicans (group [6]) and with HIC-Ca/Ct (group [1]) clear the pathogen within two weeks. A similar result is obtained with mice of sub-group [2], but CFU's corresponding to shedding of C. albicans drop more gradually. Groups inoculated with pBen016113 or with BSA (respectively [3] and, [5]) do not eliminate the pathogen effectively, as deduced from shedding numbers up to 28 days post inoculation. In other words, HIC Ca/Ct but also RC of C. albicans induce an immune response defending the host against vaginal candidiasis.

f) Course of infection of bursa upon inoculation with C. trachomatisThe result of vaginal cultures obtained at weekly intervals following intrabursal challenge, show that mice inoculated with C. trachomatis (group [7]) clear the pathogen within the first week. Mice vaccinated with HIC-Ca/Ct (group [1]) clear the pathogen within 2 weeks. Mice vaccinated with RC of C. albicans (group [2]) show a steeper reduction in IFU than groups inoculated with respectively pBen016115 or BSA (groups [4] and [5]) but take over 3 weeks to clear the pathogen. All mice of group [4] and of control groups

[5] had not fully cleared C. trachomatis from their bodies by the end of the measurement period.

In other words, HIC Ca/Ct is an effective active ingredient of a vaccine against C. trachomatis. The accelerated reduction of IFU's, albeit insufficient ability to clear C. trachomatis totally, following immunisation with Ribosomal Complex of C. albicans, illustrates the surprising immuno-stimulatory effect of Ribosomal Complex. Autopsy of mice to evaluate tissue damage to bursa, oviduct and vagina showed substantial tissue damage (necrosis and inflammation) in groups [4]and [5] but not in groups [1] and [7].

TABLE 1
GenBank sequences in the database of the National Center for
Biotechnology Information (NCBI), USA, of polynucleotide
sequences of Antigens that can be used in Polynucleotide Molecule
of the Immunogenic Complex (http://www.ncbi.nlm.nih.gov/Entrez/)

| | | NCBI |
|---|---|---|
| Organism | Antigen | accession |
| Ajellomyces dermatitidis | WI-1 adhesin | U37772 |
| Bordetella pertussis | Filamentous hemagglutinin FHA B | M60351 |
| Borrelia burgdorferi | immunodominant antigen P39 | AF116774 |
| Borrelia burgdorferi | P35 antigen | U59487 |
| bovine herpesvirus 1 | AgD, SgD & CgD antigens | AJ004801 |
| Candida albicans | Hyphal wall protein HWP 1 | U64206 |
| Chlamydia pneumoniae | 53 kDa-antigen peptide | E16639 |
| Chlamydia trachomatis | 59-kDa immunogenic protein (SK59) | M31119 |
| Chlamydia trachomatis | Major outer membrane protein (MOMP) | AF063195 |
| Escherichia coli | Colonisation factor antigen IV (CFAIV) | AJ224079.2 |
| Escherichia coli | Capsule-like surface antigen CS31 A | AF118245 to |
| | | 55 |
| Group A rotavirus | VP4 antigen | U32168 |
| Haemophilus influenzae | Hemagglutinin | U11024 |
| Haemophilus influenzae type B | Protective surface antigen D 15 | U13961 |
| Hepatitis B virus | major surface antigen (S) | AF229159 |
| Hepatitis B virus | HbcAg, HbeAg, HbsAg antigens | Z72479 |
| Hepatitis C virus | Core protein | D83645 |
| Hepatitis C virus | Core, env and E2/NS1 proteins | D50466 |
| Human herpesvirus 1 | glycoprotein G | AF120934 |
| Human herpesvirus 1 | glycoprotein B | AF259899 |
| Human immunodeficiency | gag, pol, vif, vpr, vpu, env, | U39362 |
| virus type 1 | tat, rev and nef gene products | |
| Human papilloma virus | major capsid protein L1 | V01116 |
| Human respiratory syncytial | fusion (F) protein mRNA | D00334 |
| virus | | |
| Human respiratory syncytial | attachment glycoprotein G-fusion | D00394 |
| virus | protein F gene junction | |
| Human respiratory syncytial | attachment glycoprotein G mRNA | AF065405 |
| virus strain WV2780 | | |
| Human respiratory syncytial | attachment glycoprotein G mRNA | AF065406 |
| virus strain WV5222 | | |
| Human rotavirus | inner capsid protein VP6 | AF079357 |
| Human rotavirus | VP7 | AF044357 |
| Human rotavirus | outer capsid protein VP4 | AF143408 |
| Influenza A virus | Hemagglutinin (HA) | U26830 |
| Influenza A virus | Neuraminidase (NA) | D31950 |
| Japanese encephalitis virus | prM protein, E protein | L43565 |
| Klebsiella pneumoniae | Capsule-like surface antigen CS31 A | AF118259 |
| Lymphocytic | envelope glycoprotein (GP-C) and | M20869 |
| choriomeningitis virus | nucleoprotein (NP) | |
| Measles virus | hemagglutinin | D28948 |
| Murray Valley encephalitis | Envelope protein E, precursor for | M24220 |
| virus | membrane protein (prM), non-structural | |
| | protein NS1 | |
| Mycobacterium tuberculosis | Heparin-binding hemagglutinin HBHA | AF074390 |
| Porphyromonas gingivalis | fimbrilin | D17794 |
| Pseudomonas aeruginosa | pilin of strain PAK | M14849 |
| Pseudomonas aeruginosa | Pilin of strain PAO | M11323 |
| Pseudomonas aeruginosa | Non-cytotoxic endotoxin A (mutant) | |
| Rabies virus | glycoprotein (G) | M38452 |
| Salmonella typhimurium | outer membrane porin C (ompC) | AF039309 |
| Staphylococcus aureus | Fibronectin-binding protein A (fnbA) | J04151 |
| Staphylococcus aureus | Fibronectin-binding protein b (fnbB) | X62992 |
| Streptococcus mutans | Cell surface antigen SA I/II | X17390 |
| Streptococcus parasanguis | Surface Adhesin FAB 1 | AF100426 |
| Streptococcus pneumoniae | Pneumococcal surface adhesin A | U53509 |
| Streptococcus pneumoniae | Pneumococcal surface protein PSP A | U89711 |
| Streptococcus pyogenes | Fibronectin binding protein F | AF009908 |
| Treponema denticola | Major outer sheat protein MSP | U66256 |

TABLE 2
GenBank sequences in the database of the National Center for
Biotechnology Information (NCBI), USA, of polynucleotide sequences
of vectors that can be used in Polynucleotide Molecule of the
Immunogenic Complex (http://www.ncbi.nlm.nih.gov/Entrez/)

| | | NCBI |
|---|---|---|
| Organism | Polynucleotide & function | accession |
| Escherichia coli | DNA plasmid pUC19 | X02514 |
| Home sapiens | Promoter of immunoglobulin heavy | X80347 |
| | chain | |
| Oryctolagus cuniculus | Gene & 3’flanking region of | V00882 |
| | beta-globin | |
| Semliki Forest virus | ORF1, genomic RNA, clone SFV4 | AJ251359 |

### REFERENCES

Kishimoto T., Tavassoli M., Double labelling of transferrin: tritium labelling of sialic acid and ¹²⁵I or ⁵⁹Fe labelling of the protein moiety. Anal. Biochem. 153, 324-329.

Matteucci M. D. and Caruthers M. H., Synthesis of deoxyoligonucleotides on a polymer support. J. Am. Chem. Soc., 1981, 103: 3185-3191.

Michalek S. M., and McGhee J. R., Effective immunity to dental caries: passive transfer to rats of antibodies to Streptococcs inutans elicits protection. 1977, Infect. Immun. 17: 644-650.

Pal S., Fielder T. J., Peterson E. M., de al Maza L. M., Protection against infertility in a BALB/c mouse salpingitis model by intranasal immunisation with the mouse pneumonitis biovar of Chlamydia trachomatis. Infect. Immun. 1994, 62: 3354-3362.

Youmans A. S., and Youmans G. P., Immunogenic activity of a ribosomal fraction obtained from Mycobacterium tubeculosis. 1965, J. Bacteriol. 89,1291-1298.

Yusupov M. M. and A. S. Spirin. Hot tritium bombardment technique for ribosome surface topography. Methods in Enzymology, 1988, 164: 426-439.

Sayada C., Denamur E., Orfila J., Catalan F., Elion J., Rapid genotyping of the Chlamydia trachomatis major outer membrane protein by the polymerase chain reaction. FEMS Microbiol. 1991, 83: 73-78.

Shinha N. D., Biernat J., McManus J., Koster H., Polymer support oligonucleotide synthesis XVIII: Use of β-cyanoethyl-N,Ndialkylamino/N-morpholino phosphoramidite of deoxynucleotides for the synthesis of DNA fragments simplifying deprotection and isolation of the final product. Nucleic Acids Res. 12, 1984, 12: 4539-4557.

Staehelin T., D. Maglott and R. E. Monro. Cold Spring Harbor Symp. Quant. Biol. 1969,34:39.

Tyler J. M., Branton D., J. Ultrastructural Research, 1980, 71: 95-102.

Williams & Chase, Methods of Immunology and Immunochemistry, 1967,1:155-156

1. Immunogenic Complex comprising at least one Ribosomal Complex and at least one Polynucleotide Molecule encoding Antigen of a Microbe.

2. Immunogenic Complex comprising at least one Ribosomal Complex and at least one Polynucleotide Molecule encoding Antigen, wherein Ribosomal Complex is from a Microbe and Polynucleotide encodes Antigen of virus.

3. Immunogenic Complex according to claims 1 and 2, wherein the Microbe is a bacterium.

4. Immunogenic Complex according to claims 1 and 2, wherein Microbe is a fungus.

5. Immunogenic Complex according to claims 1 and 2, wherein Microbe is a protozoa

6. Immunogenic Complex according to any one of claims 1 to 5, wherein Ribosomal Complex comprises complexes which originate from multiple Microbe species.

7. Immunogenic Complex according to any one of claims 1 to 6, wherein Polynucleotide Molecules encodes multiple Antigens.

8. Immunogenic Complex according to claims 1 to 7, comprising Ribosomal Complex which contains the large and small subunits of ribosomes in particulate form.

9. Immunogenic Complex according to claims 1 to 8, comprising Ribosomal Complex that carries minor fractions of microbial cellular membrane or cell wall components.

10. Immunogenic Complex according to claims 1 to 9, characterised in that Ribosomal Complex retains sufficient integrity to largely preserve the double-stranded nature of the large r-RNA's contained in the subunits of ribosomes.

11. Immunogenic Complex according to claims 1 to 10, wherein Polynucleotide Molecule is a DNA molecule that comprises a DNA transcription unit that encodes an Antigen, said DNA transcription unit operatively linked to regulatory sequences which control the expression of the said DNA transcription unit.

12. Immunogenic Complex according to claim 11, whereby the regulatory sequences comprise the human immunoglobulin gene control region.

13. Immunogenic Complex according to claim 11, whereby the regulatory sequences comprise the rabbit β-globin gene transcription terminator sequence.

14. Immunogenic Complex according to claim 11, whereby expression of the DNA transcription unit, that encodes an Antigen, in a host cell leads to production of a protein which is capable of inducing an immune response against said Antigen.

15. Immunogenic Complex according to claim 11, whereby expression of the DNA molecule in a host cell leads to production of a polypeptide derived or deduced or part of a protein which is capable of inducing an immune response against said Antigen.

16. Immunogenic Complex according to claims 14 and 15, wherein the host cells are eucaryotic cells belonging to vertebrate animal groups aves, Pisces and mammalia, including humans.

17. Immunogenic Complex according to claim 1 and any one of claims 3 to 16, characterised in that Ribosomal Complex and/or Polynucleotide Molecule are prepared, derived or deduced from:
(a) a bacteria selected from the group consisting of: Actinobacillus actinomycetemcomitans, Bacille Calmette-Guérin, Bordetella pertussis, Campylobacter consisus, Campylobacter recta, Capnocytophaga sp., Chlamydia trachomatis, Eikenella corrodens, Enterococcus sp., Escherichia coli, Eubacterium sp., Haemophilus influenzae, Klebsiella pneumoniae, Lactobacillus acidophilus, Listeria monocytogenes, Mycobacterium tuberculosis, Mycobacterium vaccae, Neisseria gonorrhoeae, Neisseria meningitidis, Nocardia sp., Pasteurella multocida, Porphyromonas gingivalis, Prevotella intermedia, Pseudomonas aeruginosa, Rothia dentocarius, Salmonella typhi, Salmonella typhimurium, Serratia marcescens, Shigella dysenteriae, Streptococcus mutans, Streptococcus pneumoniae, Streptococcus pyogenes, Treponema denticola, Vibrio cholera, and Yersinia enterocolitica;
(b) a fungus selected from the group consisting of Candida albicans and Blastomyces dermatitidis; or
(c) a protozoa selected from the group consisting of Plasmodium falciparum, Leishmania sp, Trypanosoma cruzi, and Entamoeba histolitica.

18. Immunogenic Complex according to claims 2 to 16, characterised in that the Ribosomal Complex is derived from bacteria under list 1 of claim 17 and that the Polynucleotide Molecule is prepared, derived or deduced from virus selected from the group consisting of:

| | |
|---|---|
| influenza virus | parainfluenza virus |
| rhinovirus | hepatitis A virus |
| hepatitis B virus | hepatitis C virus |
| apthovirus | coxsackievirus |
| rubella virus | rotavirus |
| denque virus | yellow fever virus |
| Japanese encephalitis virus | infectious bronchitis virus |
| porcine transmissible | respiratory syncytial virus |
| gastroenteric virus | |
| human immunodeficiency virus | papillomavirus |
| herpes simplex virus | varicellovirus |
| cytomegalovirus | variolavirus |
| vacciniavirus | suipoxvirus |

19. Immunogenic Complex according to claims 1 to 18, comprising Ribosomal Complex and Polynucleotide Molecule in weight ratios ranging respectively from 1 to 20 or 20 to 1.

20. Immunogenic Complex according to claims 1 to 19, characterised in that Ribosomal Complex and Polynucleotide Molecule are incorporated in a polymeric matrix.

21. Immunogenic Complex according to claims 1 to 20, whereby the polymeric matrix used, consists of chitosan-EDTA Bowman-Birk Inhibitor conjugate.

22. Immunogenic Complex according to claims 1 to 21, characterised in that Ribosomal Complex and Polynucleotide Molecule are incorporated in microparticles.

23. Immunogenic Complex according to claim 22, whereby the micro-particles used, consist of carboxymethylethylcellulose (CMEC) coated poly[dl-lactide-coglycolide] (PLG).

24. Immunogenic Complex according to claims 1 to 23, comprising Ribosomal Complex and Polynucleotide Molecule which are non-covalently coupled by ionic interactions.

25. Immunogenic Complex according to claim 24, whereby the Ribosomal Complex is covalenty conjugated to a polycation and the Polynucleotide Molecule is condensed onto said Ribosomal Complex-polycation conjugate by ionic interaction.

26. Immunogenic Complex according to claim 25 whereby the polycation used for conjugation to the Ribosomal Complex consists of poly(L-lysine).

27. Immunogenic Complex according to claim 26 whereby the average chain length of poly (L-lysine) ranges between 200 and 400 monomers.

28. Immunogenic Complex according to claims 1 to 23, comprising Ribosomal Complex and Polynucleotide Molecule which are covalently coupled.

29. Immunogenic Complex according to claim 28, whereby covalent coupling is achieved by reaction of Ribosomal Complex with 2-iminothiolate followed by addition of Polynucleotide Molecule and mild ultraviolet irradiation.

30. Immunogenic Complex according to claim 28, whereby covalent coupling is achieved by reduction of 5′thio-derivatized Polynucleotide Molecule via the freed thiol-group to maleimide-derivatized Ribosomal Complex.

31. Pharmaceutical composition for prevention and/or treatment of infectious disease caused by Microbes and/or viruses comprising Immunogenic Complex according to claims 1 to 30, wherein the Immunogenic Complex is formulated as a pharmaceutically acceptable vaccine for administration to animals and/or humans.

32. Pharmaceutical composition according claim 31 when used in prophylactic vaccines against Microbes and viruses.

33. Pharmaceutical composition according to claim 31 when used as immuno-modulator in therapeutic agents.

34. Pharmaceutical composition according to claim 31, when used as therapeutic vaccine to activate an immune response against Antigen expressed by the infectious Microbes and/or viruses during their established pathogenic phase.

35. Pharmaceutical composition according to any one of claims 31 to 34, to control whooping cough caused by Bordetella pertussis, wherein the Immunogenic Complex comprises Ribosomal Complex (IC) derived from B. pertussis, coupled to Polynucleotide Molecule (PNM) encoding the Adhesin filamentous hemaglutinin (FHA) of B. pertussis or any related protein or polypeptide derived from or corresponding to part of the fha gene product, which can still induce an antibody response FHA.

36. Pharmaceutical composition according to claims 31 to 34, to control whooping cough caused by Bordetella pertussis and respiratory tract infections caused by respiratory syncytial virus (RSV), wherein the Bacterio-viral Immunogenic Complex comprises RC derived from B. pertussis which is coupled, for a % fraction with a PNM that encodes FHA, or any related protein or polypeptide derived from or corresponding to part of FHA, which can still induce an antibody response to FHA, and for the remaining % fraction is coupled with a PNM /that encodes the fusion (F) glycoprotein (Fgp) of RSV, or any related protein or polypeptide derived from or corresponding to part of Fgp, which can still induce an antibody, response to Fgp.

37. Pharmaceutical composition according to claims 31 to 34, to control candidiasis, wherein the Heterologous Immunogenic Complex comprises RC derived from Candida albicans coupled to PNM encoding the Adhesin HWP1 of Candida albicans, or any related protein or polypeptide derived from or corresponding to part of HWP1, which can still induce an antibody response to HWP1.

38. Pharmaceutical composition according to claims 31 to 34, to control salpingitis and/or urethritis and/or cervicitis and/or trachoma, comprising RC derived from C. albicans coupled to PNM encoding Antigen SK59 of Chlamydia trachomatis, or any related protein or polypeptide derived from or corresponding to part of SK59 which can still induce an antibody response to SK59.

39. Pharmaceutical composition according to claims 31 to 34, to control candidiasis and salpingitis and/or urethritis and/or cervicitis and/or trachoma, comprising RC derived from Candida albicans which is coupled, for a % fraction with a PNM that encodes HWP1 of C. albicans, or any related protein or polypeptide derived from or corresponding to part of HWP1, which can still induce an antibody response to HWP1, and for the remaining % fraction is coupled with a PNM that encodes the SK59 protein of Chlamydia trachomatis, or any related protein or polypeptide derived from or corresponding to part of SK59 which can still induce an antibody response to SK59.

40. Use of the Immunogenic Complex according to any one of claims 1 to 30 or the pharmaceutical composition of any one of claims 31 to 39 in the preparation of a medicament for prophylaxis or treatment of infectious diseases in humans or in animals.

41. Use of the Immunogenic Complex or the pharmaceutical composition of claim 40 for prophylaxis or treatment of systemic infection and urogenital, buccal and/or ocular diseases in humans or in animals.

42. Use of the Immunogenic Complex or the pharmaceutical composition of claim 41 for prophylaxis or treatment of diseases caused by Candida sp. in humans or in animals.

43. Use of the Immunogenic Complex or the pharmaceutical composition of claims 41 for prophylaxis or treatment of diseases caused by Chlamydia sp. in humans or in animals.

44. Use of the Immunogenic Complex or the pharmaceutical composition of claim 40 for prophylaxis or treatment of respiratory diseases in humans or in animals.

45. Use of the Immunogenic Complex or the pharmaceutical composition of claim 44 for prophylaxis or treatment of diseases caused by Bordetella sp. in humans or in animals.

46. Use of the Immunogenic Complex or the pharmaceutical composition of claim 44 for prophylaxis or treatment of diseases caused by respiratory syncytial virus in humans or in animals.

47. A method of treating infectious diseases in humans or animals, or of providing prophylaxis in respect to said diseases, comprising administrating to said humans or animals an effective amount of the Immunogenic Complex of any one of claims 1 to 30 or the pharmaceutical composition of any one of claims 31 to 39.

48. A method according to claim 47 for treatment or prophylaxis of urogenital diseases.

49. A method according to claim 47 for treatment or prophylaxis of diseases caused by Candida sp., including buccal, urogenital and systemic candidiasis

50. A method according to claim 47 for treatment or prophylaxis of diseases caused by Chlamydia sp., including salpingitis, urethritis, cervicitis and trachoma.

51. A method according to claim 47 for treatment or prophylaxis of respiratory diseases.

52. A method according to claim 47 for treatment or prophylaxis of diseases caused by Bordetella sp., including whooping cough.

53. A method according to claim 47 for treatment or prophylaxis of diseases caused by respiratory syncytial virus, including lower respiratory disease.

54. A method for the manufacture of the Immunogenic Complex of any one of claims 1 to 30 comprising admixing a Ribosomal Complex with a Polynucleotide Molecule, wherein the Ribosomal complex is from a Microbe and the Polynucleotide Molecule is from, derived from or deduced from a Microbe or a virus.

55. A method for the manufacture of the Immunogenic Complex according to claim 54 whereby the Ribosomal complex and the Polynucleotide Molecule are incorporated in a polymeric matrix consisting essentially of chitosan-EDTA Bowman-Birk Inhibitor conjugate.

56. A method for the manufacture of the Immunogenic Complex according to claim 54 whereby the Ribosomal complex and the Polynucleotide Molecule are incorporated in microparticles essentially composed of carboxymethylethylcellulose-coated poly[dl-lactide-coglycolide].

57. A method for the manufacture of the Immunogenic Complex according to any one of claim 54 to 56, whereby the Ribosomal complex and the Polynucleotide Molecule are non-covalently coupled to each other, whereby the Ribosomal Complex is covalently conjugated to poly (L-lysine) and the Polynucleotide Molecule is subsequently condensed onto said Ribosomal Complex-polycation conjugate by ionic interaction.

58. A method for the manufacture of the Immunogenic Complex according to any one of claim 54 to 56, whereby the Ribosomal complex and the Polynucleotide Molecule are covalently coupled to each other by treatment of the Ribosomal Complex with 2-iminothiolate, followed by addition of Polynucleotide Molecule and mild ultraviolet irradiation.

59. A method for the manufacture of the Immunogenic Complex according to any one of claim 54 to 56, whereby the Ribosomal complex and the Polynucleotide Molecule are covalently coupled to each other by reduction of 5′ thio-derivatized Polynucleotide Molecule, via the freed thiol-group, to maleimide-derivatized Ribosomal Complex.

60. A method for the manufacture of the pharmaceutical composition of any one of claims 31 to 39 comprising admixing the Immunogenic Complex of any one of claims 1 to 30 with a pharmaceutically acceptable carrier, diluent or other excipient.

61. Methods of administration of the Immunogenic Complex according to any one of claims 1 to 30 or the pharmaceutical composition of any one of claims 31 to 39 to humans and/or animals.

62. Administration by drinking of the immunogenic Complex or of the pharmaceutical composition according to claim 61 contained in a drinkable liquid.

63. Administration by topical application of the Immunogenic Complex or the pharmaceutical composition according to claim 61 contained in a liquid solution, a gel or cream and applied to epithelial cell surfaces of infected or infection-prone areas.

64. Administration by sniffing of the Immunogenic Complex or the pharmaceutical composition according to claim 61 contained in a pernasal liquid aerosol.

65. Administration by inhalation of the Immunogenic Complex or the pharmaceutical composition according to claim 61 contained in a peroral liquid or dry powder aerosol.

66. Administration by rectal, vaginal or uteral application of the Immunogenic Complex or the pharmaceutical composition according to claim 61 contained in a suppository.
